(19) United States
(12) Patent Application Publication
(10) Pub. No.: US 2024/0252758 A1
(43) Pub. Date: Aug. 1, 2024

(54) INJECTION SYSTEM AND METHOD

(71) Applicant: CREDENCE MEDSYSTEMS, INC., Menlo Park, CA (US)

(72) Inventors: Everett Matthew Notaro, Santa Clara, CA (US); Stephen H. Diaz, Palo Alto, CA (US); Alan E. Shluzas, San Carlos, CA (US); Jeff Tillack, Foster City, CA (US)

(73) Assignee: CREDENCE MEDSYSTEMS, INC., Menlo Park, CA (US)

(21) Appl. No.: 18/428,979

(22) Filed: Jan. 31, 2024

Related U.S. Application Data
(60) Provisional application No. 63/442,175, filed on Jan. 31, 2023.

(51) Int. Cl. A61M 5/315

(52) U.S. Cl. CPC A61M 5/31511

(57) ABSTRACT

An injection system includes an injection system body defining a proximal opening at a proximal end thereof and a distal needle interface at a distal end thereof. The system also includes a stopper member and a fenestrated separator disposed in the injection system body, forming a proximal drug chamber between the stopper member and the fenestrated separator and a distal drug chamber between the fenestrated separator and the distal end of the injection system body. The system further includes a plunger member configured to be manipulated to insert the stopper member relative to the injection system body. The fenestrated separator forms an openable barrier between the proximal and distal drug chambers. The fenestrated separator is configured to allow flow from the proximal drug chamber to the distal drug chamber with increased pressure in the proximal drug chamber relative to the distal drug chamber.

The present application claims priority to U.S. Provisional Patent Application Ser. No. 63/442,175, filed on Jan. 31, 2023 under attorney docket number CM.30040.00 and entitled “INJECTION SYSTEM AND METHOD.” This application includes subject matter similar to the subject matter described in the following co-owned U.S. patent applications: (1) U.S. Utility patent application Ser. No. 14/321,706, filed Jul. 1, 2014 and issued as U.S. Pat. No. 9,814,842 on Nov. 14, 2017 under attorney docket number CM.20001.00 and entitled “SAFETY SYRINGE”; (2) U.S. Utility patent application Ser. No. 14/543,787, filed Nov. 17, 2014 and issued as U.S. Pat. No. 10,300,217 on May 28, 2019 under attorney docket number CM.20002.00 and entitled “SYSTEM AND METHOD FOR DRUG DELIVERY WITH A SAFETY SYRINGE”; (3) U.S. Utility patent application Ser. No. 14/696,342, filed Apr. 24, 2015, and issued as U.S. Pat. No. 10,010,677 on Jul. 7, 2018 under attorney docket number CM.20003.00 and entitled “SYSTEM AND METHOD FOR SAFETY SYRINGE”; (4) U.S. Utility patent application Ser. No. 15/801,239, filed on Nov. 1, 2017 and issued as U.S. Pat. No. 10,926,038 on Feb. 23, 2021 under attorney docket number CM.20011.00 and entitled “SYSTEM AND METHOD FOR SAFETY SYRINGE”; (5) U.S. Utility patent application Ser. No. 15/801,259, filed on Nov. 1, 2017, and issued as U.S. Pat. No. 10,864,330 on Dec. 15, 2020 under attorney docket number CM.20012.00 and entitled “SYSTEM AND METHOD FOR SAFETY SYRINGE”; (6) U.S. Utility patent application Ser. No. 15/801,281 filed on Nov. 1, 2017 and issued as U.S. Pat. No. 10,912,894 on Feb. 9, 2021 under attorney docket number CM.20013.00 and entitled “CARTRIDGE SAFETY INJECTION SYSTEM AND METHODS”; (7) U.S. Utility patent application Ser. No. 15/801,304 filed on Nov. 1, 2017 and issued as U.S. Pat. No. 10,960, 144 on Mar. 30, 2021 under attorney docket number CM.20015.00 and entitled “SYSTEM AND METHOD FOR SAFETY SYRINGE”; (8) U.S. Utility patent application Ser. No. 16/798,188, filed on Feb. 21, 2020 under attorney docket number CM.20023.00 and entitled “SYSTEM AND METHOD FOR SAFETY SYRINGE”; (9) U.S. Utility patent application Ser. No. 16/435,429 filed on Jun. 7, 2019 under attorney docket number CM.20019.00 and entitled “SYSTEM AND METHOD FOR SAFETY SYRINGE”; (10) U.S. Utility patent application Ser. No. 16/837,835, filed Apr. 1, 2020 under attorney docket number CM.20025.00 and entitled “POLYMERIC INJECTION SYSTEMS”; (11) U.S. Utility patent application Ser. No. 16/908,531 filed on Jun. 22, 2020 under attorney docket number CM.20026.00 and entitled “INJECTION SYSTEM AND METHOD”; (12) U.S. Utility patent application Ser. No. 17/031,108 filed on Sep. 24, 2020 under attorney docket number CM.20027.00 and entitled “SYSTEM AND METHOD FOR SAFETY SYRINGE”; (13) U.S. Provisional Patent Application Ser. No. 63/094,313 filed on Oct. 20, 2020 under attorney docket number CM.30030.00 and entitled “RETRACTION MECHANISM FOR SAFE INJECTION SYSTEM”; (14) U.S. Utility patent application Ser. No. 16/435,429, filed on Jun. 7, 2019 under attorney docket number CM.20019.00 and entitled “SYSTEM AND METHOD FOR SAFETY SYRINGE”; (15) U.S. Provisional Patent Application Ser. No. 62/729,880, filed on Sep. 11, 2018 under attorney docket number CM.30021.00 and entitled “SYSTEM AND METHOD FOR SAFETY SYRINGE”; (16) U.S. Utility patent application Ser. No. 17/364,546, filed on Jun. 30, 2021 under attorney docket number CM.20028.00 and entitled “SYSTEM AND METHOD FOR SAFETY SYRINGE”; (17) U.S. Provisional Patent Application Ser. No. 63/156,264, filed on Mar. 3, 2021 under attorney docket number CM.30031.00 and entitled “SYSTEM AND METHOD FOR SAFETY SYRINGE”; (18) U.S. Provisional Patent Application Ser. No. 63/193,466, filed on May 26, 2021 under attorney docket number CM.30032.00 and entitled “SYSTEM AND METHOD FOR SAFETY SYRINGE”; (19) U.S. Utility patent application Ser. No. 18/098,295, filed on Jan. 18, 2023 under attorney docket number CM.20034.00 and entitled “INJECTION SYSTEM AND METHOD”; (20) U.S. Provisional Patent Application Ser. No. 63/414,055, filed on Oct. 7, 2023 under attorney docket number CM.30039.00 and entitled “INJECTION SYSTEM AND METHOD”; and (21) U.S. Utility patent application Ser. No. 18/377,601, filed on Oct. 6, 2023 under attorney docket number CM.20039.00 and entitled “INJECTION SYSTEM AND METHOD”. The contents of the patent applications and patents identified herein are fully incorporated herein by reference as though set forth in full.

### FIELD OF THE DISCLOSURE

The present disclosure relates generally to injection systems, devices, and processes for facilitating various levels of control over fluid infusion, and more particularly to systems and methods related to serial injection and mixing liquids before injection, with or without safety features, in healthcare environments.

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle, vial, bag, or other drug containment system (10). Indeed, due to regulatory constraints in some countries such as the United States as well as sterility maintenance concerns, upon use of a medicine bottle (10) with a syringe (2) as shown in a particular patient's environment, such medicine bottle may only be utilized with a single patient and then must be disposed of—causing significant medical waste from bottle and remaining medicine disposal, and even contributing to periodic shortages of certain critical drugs. Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer manifold assembly of FIG. 2B may be used to administer liquid drugs to the patient intravenously with or without the use of an intravenous infusion bag. The Luer fittings (14) of the syringes of FIG. 2A may be termed the “male” Luer fittings, while those of FIG. 2B (18) may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting (14) which are configured to engage a flange on the female fitting (18) and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during assembly of a Luer coupling. The use of needle injection configurations, on the other hand, carries with it the risk of a sharp needle contacting or stabbing a person or structure that is not desired. For this reason, so called “safety syringes” have been developed.

One embodiment of a safety syringe (20) is shown in FIG. 3, wherein a tubular shield member (22) is spring biased to cover the needle (8) when released from a locked position relative to the syringe body (4). Another embodiment of a safety syringe (24) is shown in FIGS. 4A-4B. With such a configuration, after full insertion of the plunger (6) relative to the syringe body (4), the retractable needle (26) is configured to retract (28, 26) back to a safe position within the tubular body (4), as shown in FIG. 4B. Such a configuration which is configured to collapse upon itself may be associated with blood spatter/aerosolization problems, the safe storage of pre-loaded energy which may result in possible malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for prefilled syringe assemblies such as those depicted in FIGS. 5A and 5B, which generally comprise a syringe body, or “drug enclosure containment delivery system”, (34), a plunger tip, plug, or stopper (36), and a distal seal or cap (35) which may be fitted over a Luer type interface (FIG. 5A shows the cap 35 in place; FIG. 5B has the cap removed to illustrate the Luer interface 14). Liquid medicine may reside in the volume, or medicine reservoir, (40) between the distal seal and the distal end (37) of the plunger tip (36). The plunger tip (36) may comprise a standard butyl rubber material and may be coated, such as with a biocompatible lubricious coating (e.g., polytetrafluoroethylene (“PTFE”)), to facilitate preferred sealing and relative motion characteristics against the associated syringe body structure and material. The proximal end of the injection system body (34) in FIG. 5B comprises a conventional integral syringe flange (38), which is formed integral to the material of the injection system body (34). The flange (38) is configured to extend radially from the injection system body (34) and may be configured to be a full circumference, or a partial circumference around the injection system body (34). A partial flange is known as a “clipped flange” while the other is known as a “full flange.” The flange is used to grasp the syringe with the fingers to provide support for pushing on the plunger to give the injection. The injection system body (34) preferably comprises a translucent material such as a glass or polymer. To form a contained volume within the chamber or reservoir (40), and to assist with expulsion of the associated fluid through the needle, a plunger tip (36) may be positioned within the injection system body (34). The injection system body (34) may define a substantially cylindrical shape (i.e., so that a plunger tip 36 having a circular cross-sectional shape may establish a seal against the injection system body (34)), or be configured to have other cross-sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus certain suppliers have moved to more “vertical” solutions, such as that (41) featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Moreover, an increasing number of injectable liquids (e.g., medicines) have an additional requirement that two or more components are preferably injected serially (e.g., into a patient) within a short time (e.g., seconds) of each other. Multiple components can be injected serially using separate injection devices (e.g., pre-loaded syringes) or using the same injection device to serially draw the multiple components from separate open containers and serially inject them. However, such serial injection using separate injection devices or serially drawing and injecting the multiple components necessarily results in multiple needle insertions into a patient, and can be inaccurate and lead to loss of components. Further, serial injection using separate injection devices or serially drawing the multiple components into a syringe can lead to unnecessary exposure of a user to one or more uncapped needles. Moreover, serial injection using separate injection devices or serially drawing and injecting the multiple components can cause an unacceptable lag between injections of the multiple components. Existing serial injection systems utilize transfer tubes with various openings that cooperate with various stopper members to facilitate serial injection. However, the various openings may become clogged and errors in the complex cooperation between the openings and stopper members may result in injection errors.

In some cases there may be a chemical reaction that takes place between the components of a multi-component injection. The use of traditional style dual chamber syringes, such as disclosed in U.S. Utility patent application Ser. Nos. 14/696,342 and 17/364,546, which were previously incorporated by reference herein, where the components are mixed together inside of the syringe, may not be compatible with these reactive components as the mixed components may not be suitable for injection. Some illustrative examples of this phenomenon include when the components are mixed together, the viscosity of the combined medicine increases such that the medicine cannot be easily injected through a needle. Also, other reactions such as exothermic, endothermic, etc. may take place that could prevent the use of a traditional dual chamber injection system.

In addition, an increasing number of injectable liquids (e.g., medicines) have yet another requirement that time of exposure of the injectable liquid to metals (e.g., stainless steel of a needle) be minimized. Still another requirement is the desirability of systems suitable for patient self-injection.

It is also desirable to incorporate needle stick prevention technology into the injection system. The ability to retract the sharp end of the needle at least partially inside of the syringe protects the person giving the injection and the patient from inadvertent needle stick injuries.

Exemplary safe injection systems include needle retraction systems such as those described in U.S. patent application Ser. Nos. 14/696,342, 14/543,787, 14/321,706, 15/801,239, 15/801,259, 15/801,281, 15/801,304, 16/908,531, 17/031,108, and 63/094,313, the contents of which have been previously Incorporated by reference herein. Retracting the needle assembly moves the sharp needle distal end inside of the needle hub or the injection system body/syringe body to prevent accidental needle sticks.

Similar problems exist in known systems for mixing two or more liquids before injecting the mixed liquid.

There is a need for injection systems that address the shortcomings of currently-available configurations. In particular, there is a need for multiple chamber serial injection, and liquid mixing and injection solutions with precise control, which may utilize the existing and relatively well-controlled supply chain of conventionally delivered prefilled syringe assemblies such as those described in reference to FIGS. 5A and 5B.

### SUMMARY

Embodiments are directed to injection systems. In particular, the embodiments are directed to serial injection, and liquid mixing and injection systems with precise control of handling, mixing, and delivery of serial injectables.

In one embodiment, an injection system includes an injection system body defining a proximal opening at a proximal end thereof and a distal needle interface at a distal end thereof. The system also includes a stopper member and a fenestrated separator disposed in the injection system body, forming a proximal drug chamber between the stopper member and the fenestrated separator and a distal drug chamber between the fenestrated separator and the distal end of the injection system body. The system further includes a plunger member configured to be manipulated to insert the stopper member relative to the injection system body. The plunger may be manually manipulated by a user or manipulated by a device such as an auto-injector or a pen injector. The fenestrated separator forms an openable barrier between the proximal and distal drug chambers. The fenestrated separator is configured to allow flow from the proximal drug chamber to the distal drug chamber with increased pressure in the proximal drug chamber relative to the distal drug chamber.

In one or more embodiments, the fenestrated separator is configured to move longitudinally within the injection system body. The fenestrated separator may include proximal and distal gaskets disposed adjacent respective proximal and distal ends thereof and extending radially therefrom. The fenestrated separator may be configured to maintain contact between an inner surface of the injection system body and the proximal gasket and between the inner surface of the injection system body and the distal gasket as the fenestrated separator moves longitudinally within the injection system body. The proximal and distal gaskets may be separated by a minimum longitudinal distance.

In one or more embodiments, the proximal and distal circumferential gaskets is configured to form respective first and second fluid-tight seals with an inner surface of the injection system body. The proximal and distal circumferential gaskets may be made from an elastic material. The elastic material may be rubber, thermoplastic elastomer, butyl rubber, or polyisoprene elastomer. The fenestrated separator may include a port configured to be opened by the increased pressure in the proximal drug chamber relative to the distal drug chamber.

In one or more embodiments, the fenestrated separator includes a rigid portion and an elastic portion. The elastic portion may define a pocket configured to receive the rigid portion therein. The rigid portion may be made from cyclic olefin copolymer. The elastic portion may be made from rubber, thermoplastic elastomer, butyl rubber, or polyisoprene elastomer.

In one or more embodiments, the elastic portion includes an annular flap configured to be disposed adjacent a distal surface of the rigid portion. The elastic portion also includes a ring configured to support the annular flap. The elastic portion further includes proximal and distal circumferential gaskets extending from an outer circumference of the ring. The proximal and distal circumferential gaskets may be configured to form respective first and second fluid-tight seals with an inner surface of the injection system body. The rigid portion may include an annular portion. The annular portion may define a port extending therethrough. The port may be configured to be removably closed by the annular flap of the elastic portion. The annular flap of the elastic portion may be configured such that the annular flap is biased to removably close the port, and the increased pressure in the proximal drug chamber relative to the distal drug chamber deforms the annular flap away from the port defined by the annular portion of the rigid portion, thereby opening the port.

In one or more embodiments, the annular portion defines a plurality of ports extending therethrough. Each of the plurality of ports may be configured to be removably closed by the annular flap of the elastic portion. The annular flap of the elastic portion may be configured such that the annular flap is biased to removably close each of the plurality of ports, and the increased pressure in the proximal drug chamber relative to the distal drug chamber deforms the annular flap away from each of the plurality of ports defined by the annular portion of the rigid portion, thereby opening each of the plurality of ports.

In one or more embodiments, the rigid portion includes a distally extending portion coupled to an annular portion. The elastic portion may have a radially inward circumferential surface defining a central port. The radially inward circumferential surface of the elastic portion may be configured to form a fluid-tight seal around the distally extending portion of the rigid portion, thereby closing the central port in the elastic portion. The elastic portion may be biased to close the central port therein with the distally extending portion of the rigid portion. The increased pressure in the proximal drug chamber relative to the distal drug chamber may deform the elastic portion away from the distally extending portion of the rigid portion, removing the fluid-tight seal therearound, and opening the central port in the elastic portion. The distally extending portion of the rigid portion may define a circumferential groove configured to receive the radially inward circumferential surface of the elastic portion.

In one or more embodiments, the rigid portion defines a first port, and the elastic portion defines a second port. The fenestrated separator may have a closed configuration and an open configuration. In the closed configuration, both the first and second ports are closed, and in the open configuration, both the first and second ports are open. In the open configuration a flow path may fluidly couple the proximal and distal drug chambers through the first and second ports. The elastic portion may be biased to close the first and second ports, thereby placing the fenestrated separator in the closed configuration. The fenestrated separator may be configured to transform from the closed configuration to the open configuration with increased pressure in the proximal drug chamber relative to the distal drug chamber.

In another embodiment, a method for injecting includes providing a prefilled injection system. The injection system includes an injection system body defining a proximal opening at a proximal end thereof and a distal needle interface at a distal end thereof. The injection system also includes a stopper member and a fenestrated separator disposed in the injection system body, forming a proximal drug chamber between the stopper member and the fenestrated separator and a distal drug chamber between the fenestrated separator and the distal end of the injection system body. The injection system further includes a first drug and an air bubble disposed in the distal drug chamber. Moreover, the injection system includes a second drug disposed in the proximal drug chamber. In addition, the injection system includes a plunger member coupled to the stopper member. The method also includes agitating the prefilled injection system to mix the first drug. The method further includes attaching a needle assembly with a needle to the distal needle interface at the distal end of the injection system body. Moreover, the method includes positioning the injection system body such that the needle is pointed upward. In addition, the method includes moving the plunger and the stopper member coupled thereto distally into the injection system body with the needle pointed upward to remove at least a portion of the air bubble from the distal drug chamber. The method also includes moving the plunger and the stopper member coupled thereto distally further into the injection system body to eject at least a portion of the first drug from the distal drug chamber through the needle and move the fenestrated separator longitudinally toward the distal end of the injection system body, and move the plunger and the stopper member coupled thereto distally still further into the injection system body to open the fenestrated separator and transfer at least a portion of the second drug from the proximal drug chamber to the distal drug chamber. In addition, the method includes moving the plunger and the stopper member coupled thereto distally yet further into the injection system body to eject the at least a portion of the second drug from the distal drug chamber through the needle.

In one or more embodiments, moving the plunger and the stopper member coupled thereto distally still further into the injection system body after the at least a portion of the first drug has been ejected from the distal drug chamber through the needle raises the pressure in the proximal drug chamber relative to the distal drug chamber, thereby opening the fenestrated separator. The method may also include moving the fenestrated separator into contact with a distal end of the injection system body, and moving the stopper member into contact with the fenestrated separator, thereby completing an injection.

In another embodiment, an injection system includes an injection system body defining a proximal opening at a proximal end thereof and a distal needle interface at a distal end thereof. The system also includes a proximal stopper member, a middle fenestrated member, and a distal stopper member disposed in the injection system body. The proximal stopper member, the middle fenestrated member, the distal stopper member, and the injection system body form a proximal drug chamber between the proximal stopper member and the middle fenestrated separator, a middle drug chamber between the middle fenestrated separator and the distal stopper member, and a distal drug chamber between the distal stopper member and the distal end of the injection system body. The system further includes a plunger member configured to be manipulated to insert the proximal stopper member relative to the injection system body. The plunger may be manually manipulated by a user or manipulated by a device such as an auto-injector or a pen injector. The fenestrated separator forms an openable barrier between the proximal and middle drug chambers. The fenestrated separator is configured to selectively allow flow from the proximal drug chamber to the middle drug chamber.

In one or more embodiments, the middle fenestrated separator is configured to move longitudinally within the injection system body. The middle fenestrated separator may include proximal and distal gaskets disposed adjacent respective proximal and distal ends thereof and extending radially therefrom. The middle fenestrated separator may be configured to maintain contact between an inner surface of the injection system body and the proximal gasket and between the inner surface of the injection system body and the distal gasket as the middle fenestrated separator moves longitudinally within the injection system body. The proximal and distal gaskets may be separated by a minimum longitudinal distance.

In one or more embodiments, the proximal and distal circumferential gaskets are configured to form respective first and second fluid-tight seals with an inner surface of the injection system body. The proximal and distal circumferential gaskets may be made from an elastic material. The elastic material may be rubber, thermoplastic elastomer, butyl rubber, or polyisoprene elastomer.

In one or more embodiments, the middle fenestrated separator defines a middle port, and the middle fenestrated separator includes a plug removably disposed in the middle port and configured to seal the middle port. The injection system may also include a needle having a needle proximal end, and the plug may be configured to be dislodged from the middle port by the needle proximal end. The middle fenestrated separator may define a distally facing funnel configured to guide the needle proximal end to the plug in the middle port.

The aforementioned and other embodiments of the invention are described in the Detailed Description which follows.

### BRIEF DESCRIPTION OF THE DRAWINGS

This patent or application file contains at least one drawing executed in color. Copies of this patent or patent application publication with color drawings will be provided by the U.S. Patent and Trademark Office upon request and payment of the necessary fee.

FIGS. 1A to 5C illustrate various aspects of conventional injection syringe configurations.

FIGS. 6 and 7 are perspective and exploded views depicting a dual chamber serial injection system, according to some embodiments.

FIG. 8 is a perspective view depicting a prefilled dual chamber serial injection system\, according to some embodiments.

FIGS. 9A and 9B are perspective and exploded views depicting a fenestrated separator, according to some embodiments.

FIGS. 10A to 10D are side (FIG. 10A), side cross-sectional (FIG. 10B), left/distal (FIG. 10C), and right/proximal (FIG. 10D) views depicting an elastic portion of a fenestrated separator, according to some embodiments.

FIGS. 11A to 11D are side (FIG. 11A), left/distal (FIG. 11B), right/proximal (FIG. 11C), and side cross-sectional (FIG. 11D) views depicting a rigid portion of a fenestrated separator, according to some embodiments.

FIG. 12 is a side cross-sectional view depicting a fenestrated separator (900) in a closed configuration.

FIGS. 13 and 14 are side cross-sectional and perspective views of a fenestrated separator in an open configuration, according to some embodiments.

FIGS. 15A to 17B are detailed side cross-sectional views depicting a fenestrated separator opening and closing in a serial injection system, according to some embodiments.

FIGS. 18 to 29 are side cross-sectional views depicting various steps in a serial injection method using a dual chamber serial injection system, according to some embodiments.

FIGS. 30A to 30D schematically depict various steps in a serial injection method using a dual chamber serial injection system, according to some embodiments.

FIGS. 31 to 38 are side (FIGS. 31 and 35 to 38) and side cross-sectional (FIGS. 32 to 34) views depicting various steps in a liquid mixing and injection method using a triple chamber injection system, according to some embodiments.

FIG. 39 is a detailed side view of a triple chamber injection system, according to some embodiments.

FIGS. 40 to 42 are detailed views of a middle fenestrated separator for use with a triple chamber injection system, according to some embodiments.

FIGS. 43 and 44 are side cross-sectional views of a fenestrated separator in a closed (FIG. 43) and an open (FIG. 44) configuration, according to some embodiments.

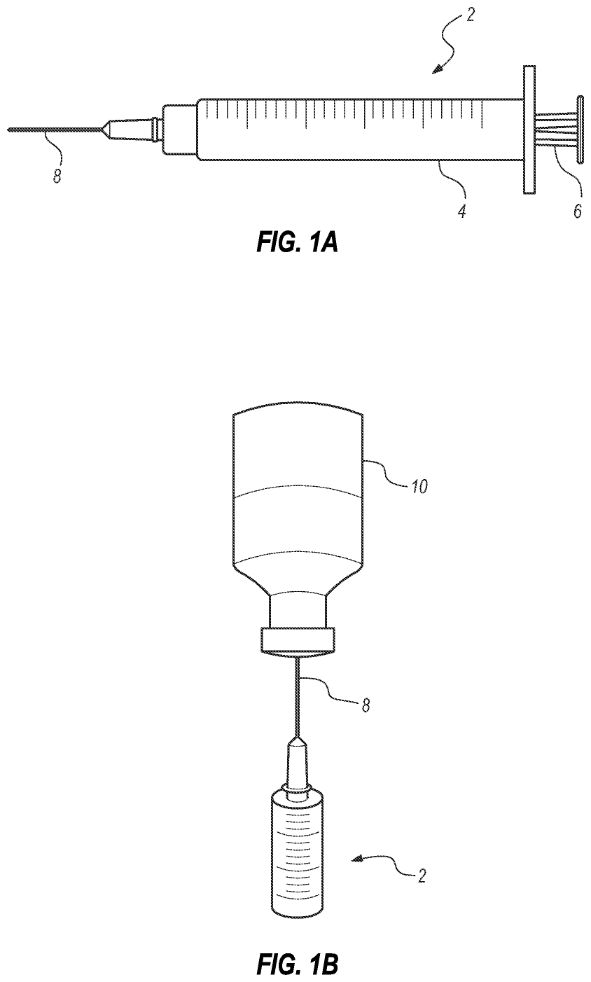

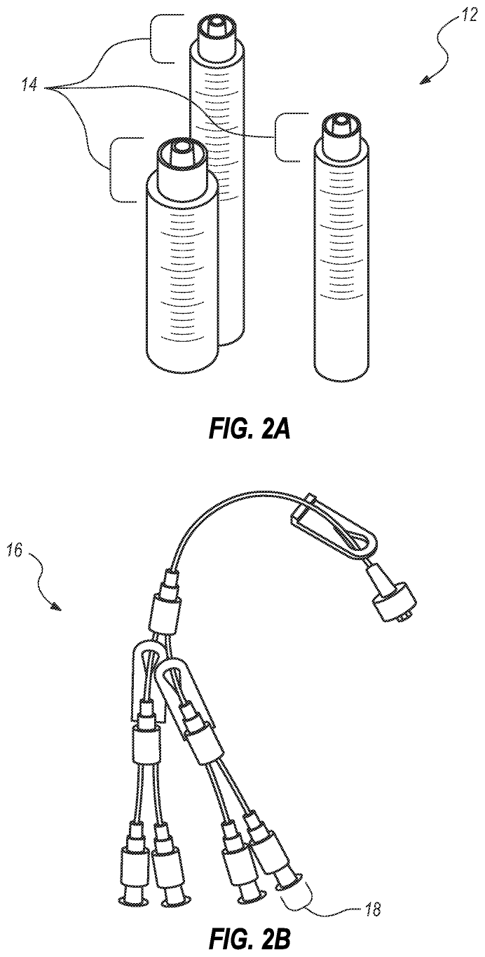

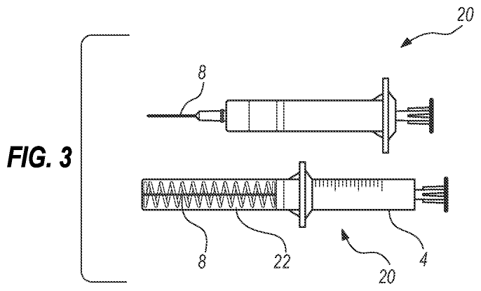

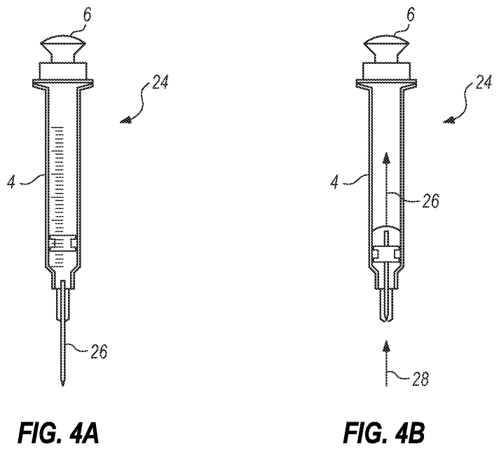

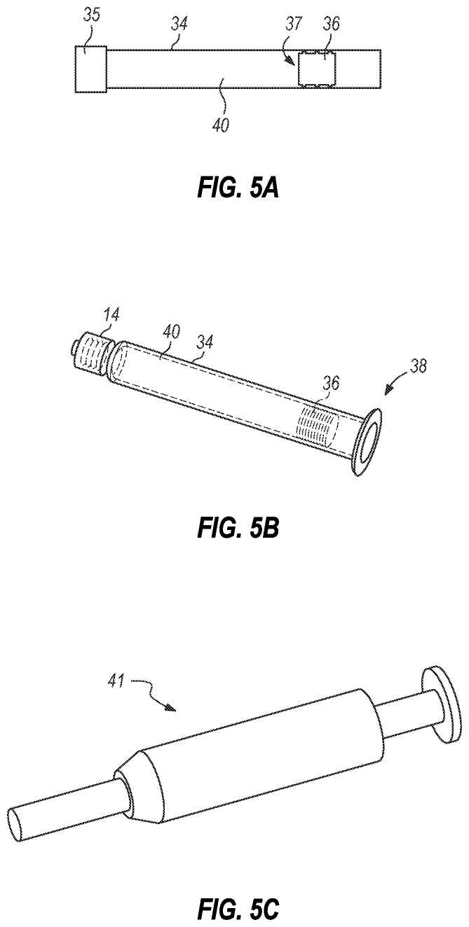

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

### Exemplary Multi-Chamber Serial Injection Systems and Method

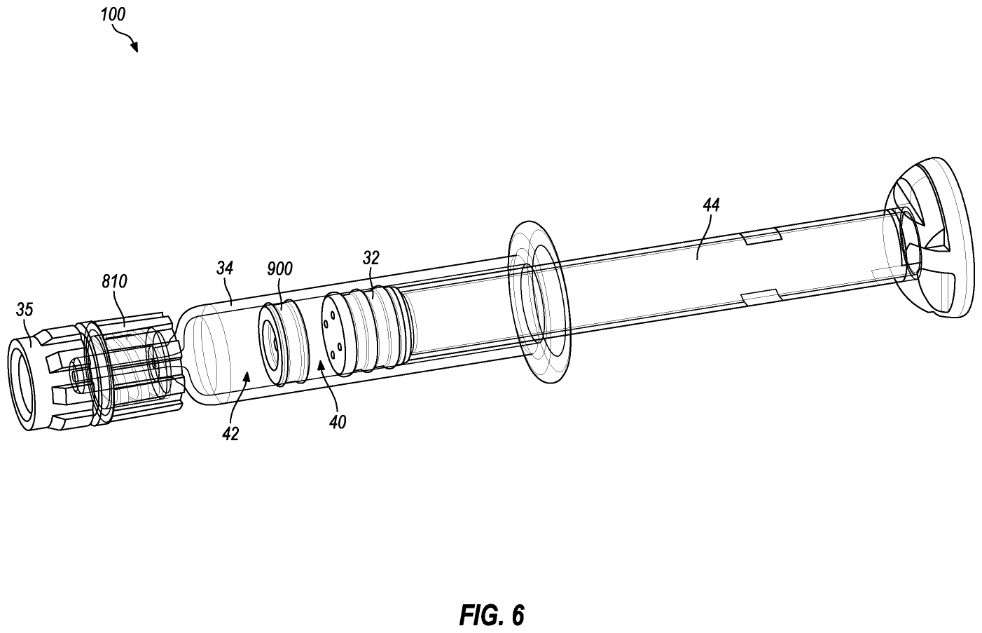

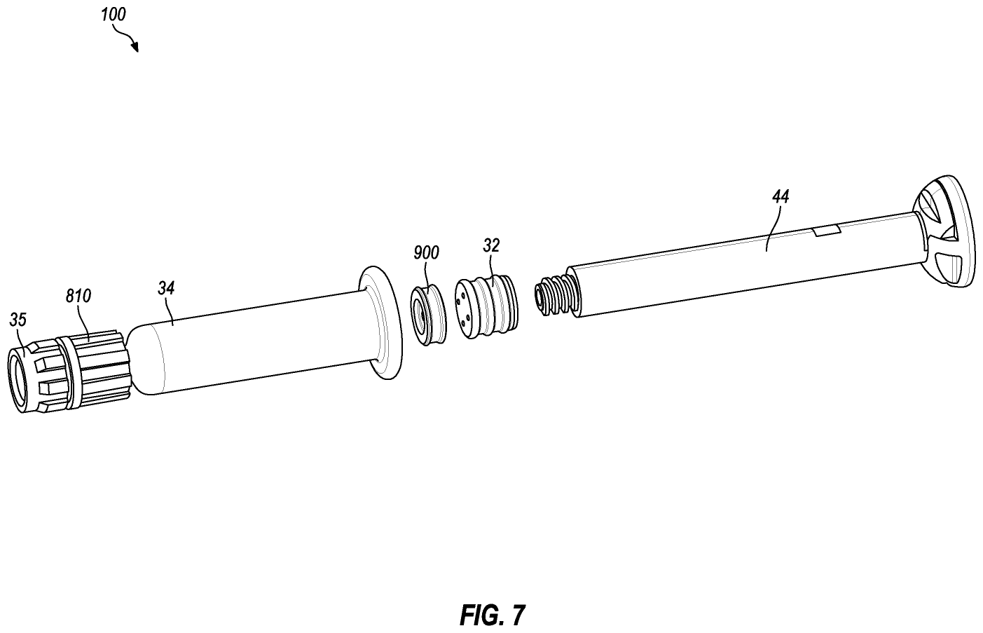

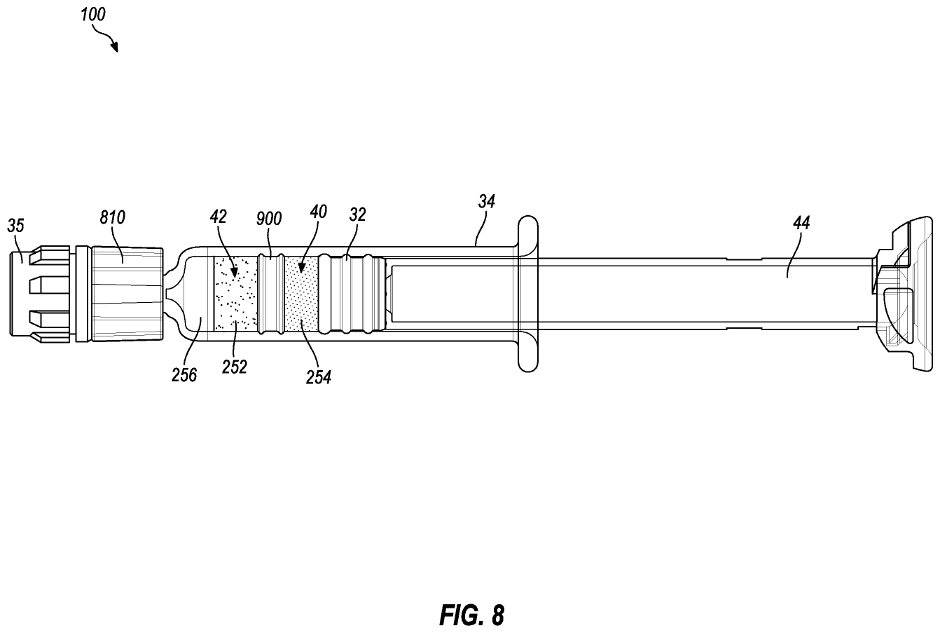

FIGS. 6 and 7 are a perspective view and an exploded view depicting a dual chamber serial injection system (100), according to some embodiments. FIG. 8 is a perspective view of a dual chamber serial injection system (100) that is prefilled with first and second liquids (252, 254), which may be first and second drugs for sequential injection, according to some embodiments. The dual chamber serial injection system (100) includes a conventional off-the-shelf injection system body (34) with a conventional off-the-shelf stopper member (32) disposed therein. The dual chamber serial injection system (100) also includes a fenestrated separator (900) disposed in the injection system body (34) at a distance distal of the stopper member (32). The stopper member (32) and the fenestrated separator (900) together with the injection system body (34) define proximal and distal drug chambers (40, 42). First and second liquids (252, 254) are contained in the distal and proximal drug chambers (42, 40), respectively (see FIG. 8). The stopper member (32) and the fenestrated separator (900) occlude the proximal and distal ends of the proximal drug chamber (40). The fenestrated separator (900) occludes a proximal end of the distal drug chamber (42). In some embodiments, the distal surface of the stopper member (32) and a proximal surface of the fenestrated separator (900) are coated with a lubricious polymer coating (e.g., PTFE or ETFE), the polymer coatings of the stopper member (32) and the fenestrated separator (900), together with the injection system body (34) define the proximal drug chamber (40). The lubricious polymer coating also serves to isolate the rubber of the stopper member (32) and the fenestrated separator (900) from the second liquid (254). In other embodiments, the fenestrated separator (900) may be constructed from un-coated butyl rubber such as chlorobutyl and/or bromobutyl rubber. Alternatively, the fenestrated separator (900) may be constructed of other elastomeric materials such as TPE, TPU, silicone rubber, or other elastomers.

The dual chamber serial injection system (100) further includes a plunger member (44) coupled to the stopper member (32) and configured to insert the stopper member (32) distally into the injection system body (34).

The dual chamber serial injection system (100) facilitates serial injection of the first liquid (252) from the distal drug chamber (42) followed by injection of the second liquid (254) from the proximal drug chamber (40) subject to serial insertion of a plunger assembly (44) relative to the injection system body (34) to various degrees by a user. The plunger assembly (44) includes a plunger housing member (69) coupled to the stopper member (32) and a plunger manipulation interface (128). The plunger manipulation interface (128) may be configured to be pushed by an operator or may be configured to interface with a device such as an auto injector or a pen injector. In some embodiments including an auto-injector, a linear motor or one or more compressed springs are configured to apply distally directed and/or proximally directed forces to the plunger manipulation interface to manipulate the plunger assembly (44). The first and second liquids (252, 254) located in the distal and proximal drug chambers (42, 40) respectively may be any liquid or gel, such as aqueous or oil-based medicine solutions.

The injection system body (34) includes a distal needle interface (810) at a distal end thereof. In some embodiments, the distal needle interface (810) may be female Luer connector. The dual chamber serial injection system (100) also includes a distal seal or cap (35), which is removably coupled to the distal needle interface (810) to fluidly seal the distal needle interface (810). While the dual chamber serial injection system (100) is depicted as having a distal needle interface (810), in other embodiments, a staked needle mounted on a distal end of the injection system body (34). While the injection system body (34) depicted in FIGS. 6 to 8 is a syringe body, the injection system body (34) may be a cartridge body instead of a syringe. Such cartridge injection systems are disclosed in U.S. Utility patent application Ser. No. 15/801,281, which was previously incorporated by reference herein.

### 1. Fenestrated Separators

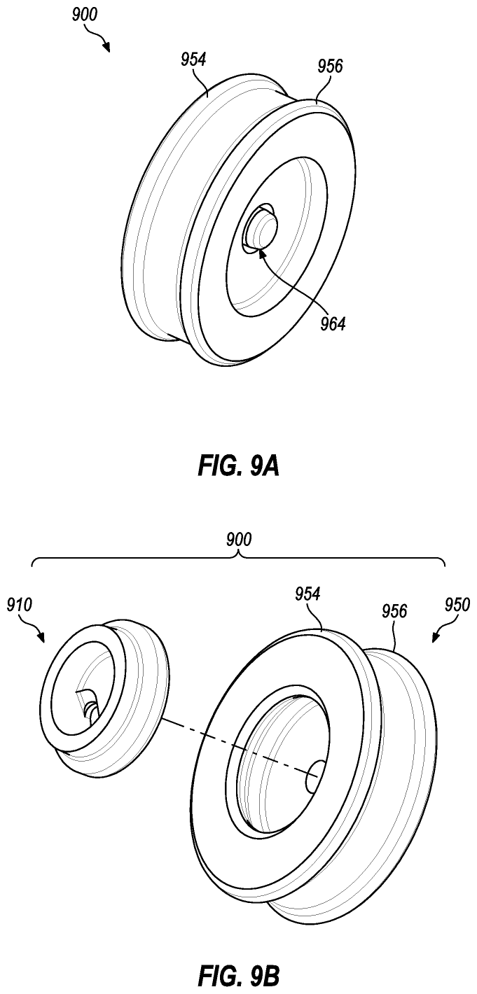

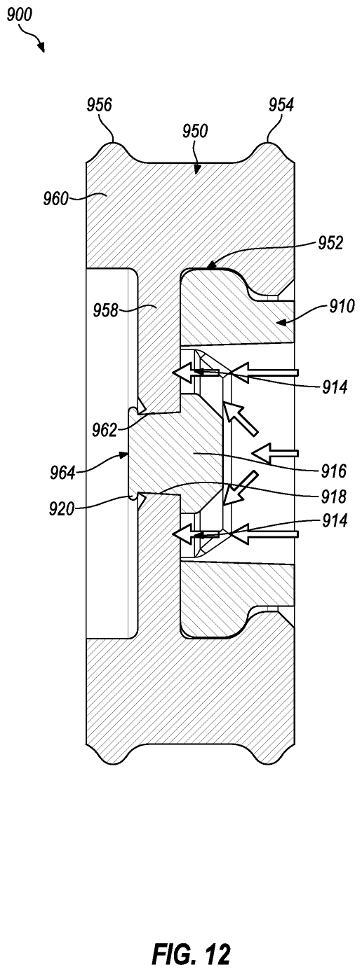

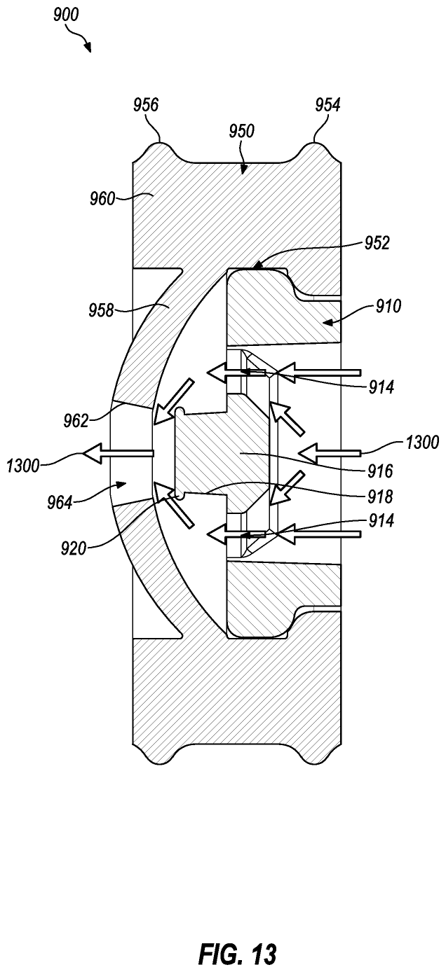

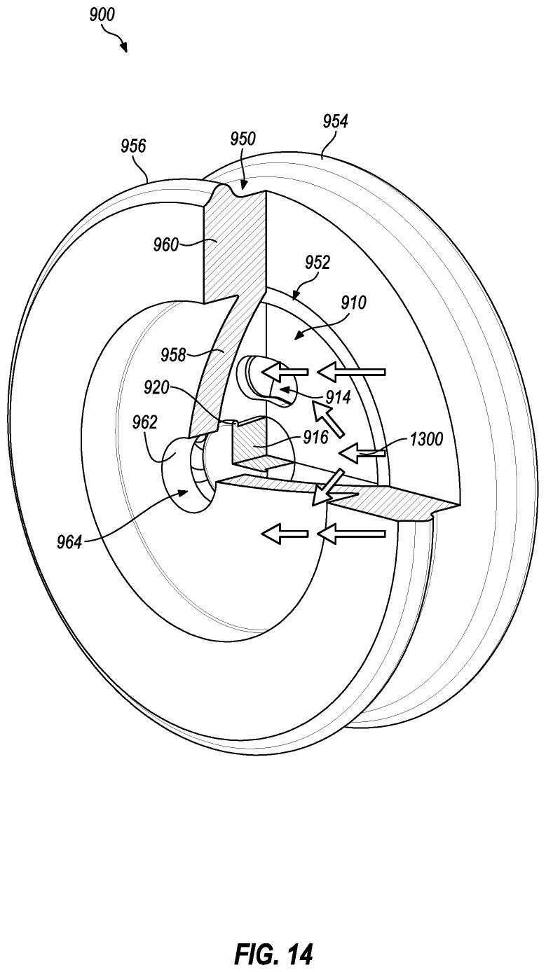

FIGS. 9A to 12 depict a fenestrated separator (900) for use with dual chamber serial injection systems (100), according to some embodiments. The fenestrated separator (900) is configured to be disposed in the injection system body (34) to define the proximal and distal drug chambers (40, 42) as described above. The fenestrated separator (900) has a closed configuration (see FIG. 12), in which fluid flow through the fenestrated separator (900) is prevented, and an open configuration (see FIGS. 13 and 14), in which fluid is allowed to flow from the proximal drug chamber (40) to the distal drug chamber (42) through the fenestrated separator (900). The fenestrated separator (900) is configured to change from the closed configuration to the open configuration with increased pressure in the proximal drug chamber (40) relative to the distal drug chamber (42).

FIGS. 9A and 9B are perspective and exploded views of a fenestrated separator (900), according to some embodiments. The fenestrated separator (900) includes a rigid portion (910) disposed within an elastic portion (950). The elastic portion (950) defines a pocket (952) in which the rigid portion (910) is disposed. The rigid portion (910) may be made from a polymer such as cyclic olefin copolymer (COC) and/or a cyclic olefin polymer (COP). Alternatively, the rigid portion (910) may be made of glass, ceramic, metal, or other non-reactive material with the drug product in the distal drug chamber (42). The elastic portion (950) may be made from rubber, thermoplastic elastomer (TPE), butyl rubber such as chlorobutyl or bromobutyl, silicone rubber, and/or polyisoprene elastomer.

The fenestrated separator (900) is also configured to move longitudinally within the injection system body (34). The elastic portion (950) of the fenestrated separator (900) includes a proximal gasket (954) and a distal gasket (956). The proximal and distal gaskets (954, 956) are annular bodies that extend radially outward from respective proximal and distal ends of the elastic portion (950) of the fenestrated separator (900). The proximal and distal gaskets (954, 956) are made from elastic materials and are configured to form first and second fluid-tight seals with an inner surface of the injection system body (34). The elastic materials may be rubber, thermoplastic elastomer (TPE), butyl rubber, silicone rubber, or polyisoprene elastomer. The fenestrated separator (900) may be PTFE coated on the surfaces facing one or both of the proximal and distal drug chambers (40, 42).

While the fenestrated separator (900) moves longitudinally within the injection system body (34), the fenestrated separator (900) is configured to maintain contact between the inner surface of the injection system body (34) and both of the proximal and distal gaskets (954, 956). To that end, the proximal and distal gaskets (954, 956) are separated by a minimum longitudinal distance. Maintaining contact between the inner surface of the injection system body (34) and both of the proximal and distal gaskets (954, 956) prevents the fenestrated separator (900) from twisting or flipping about a diameter thereof, thereby maintaining the respective first and second fluid-tight seals.

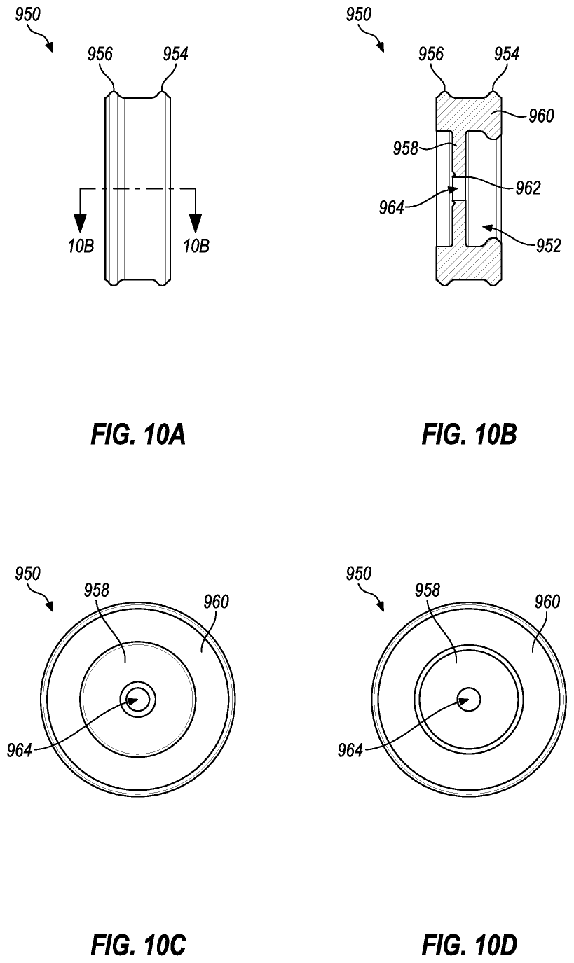

FIGS. 10A to 10D are side (FIG. 10A), side cross-sectional (FIG. 10B), left/distal (FIG. 10C), and right/proximal (FIG. 10D) views of an elastic portion (950) of a fenestrated separator (900), according to some embodiments. As described above and shown in FIG. 10B, the elastic portion (950) defines a pocket (952) in which the rigid portion (910) is disposed. The proximal and distal gaskets (954, 956) are also shown in FIGS. 10A and 10B. The elastic portion (950) of the fenestrated separator (900) also defines an annular flap (958) supported by a ring (960) as shown in FIGS. 10B, 10C, and 10D. As shown in FIG. 10B the annular flap (958) of the elastic portion (950) has a radially inward circumferential surface (962) that defines a central port (964) through the annular flap (958).

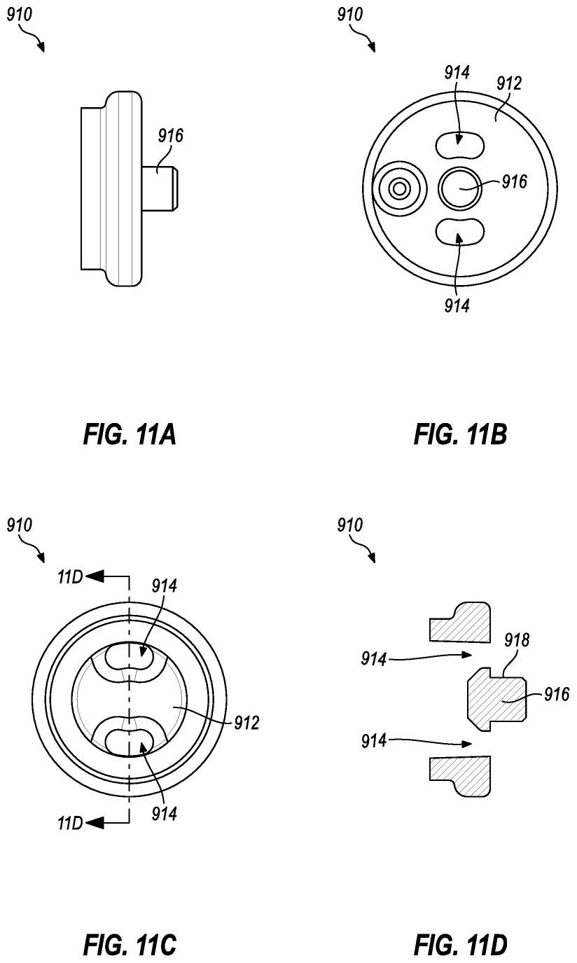

FIGS. 11A to 11D are side (FIG. 11A), left/distal (FIG. 11B), right/proximal (FIG. 11C), and side cross-sectional (FIG. 11D) views of a rigid portion (910) of a fenestrated separator (900), according to some embodiments. The rigid portion (910) of the fenestrated separator (900) defines an annular portion (912) that defines a pair of ports (914) therethrough as shown in FIGS. 11B, 11C, and 11D. When the rigid portion (910) is disposed in the pocket (952) in the elastic portion (950), the annular flap (958) of the elastic portion (950) is biased to fluidly seal/close the ports (914) in the annular portion (912) of the rigid portion (910). As shown in FIGS. 11A, 11B, and 11D, the rigid portion (910) also defines a distally extending portion (916) that extends distally from an approximate center of the distal surface of the annular portion (912) of the rigid portion (910). The distally extending portion (916) defines a circumferential groove (918) configured to receive the radially inward circumferential surface (962) of the annular flap (958). When the rigid portion (910) is disposed in the pocket (952) in the elastic portion (950), the radially inward circumferential surface (962) of the annular flap (958) of the elastic portion (950) is biased to form a fluid-tight seal around the distally extending portion (916) of the rigid portion (910) to fluidly seal/close the central port (964) in the annular flap (958) of the elastic portion (950).

FIG. 12 is a side cross-sectional view of a fenestrated separator (900) in a closed configuration, according to some embodiments. In the closed configuration, the ports (914) in the annular portion (912) of the rigid portion (910) are fluidly sealed/closed by the annular flap (958) of the elastic portion (950). Similarly, in the closed configuration, the central port (964) in the annular flap (958) of the elastic portion (950) is fluidly sealed/closed by the distally extending portion (916) of the rigid portion (910). As such, fluid flow from the proximal drug chamber (40) to the distal drug chamber (42) is prevented by the fenestrated separator (900) in the closed configuration.

FIGS. 13 and 14 are side cross-sectional and perspective views of a fenestrated separator (900) in an open configuration, according to some embodiments. In the open configuration, increased pressure in the proximal drug chamber (40) relative to the distal drug chamber (42) deforms/domes the annular flap (958) of the elastic portion (950) distally away from the annular portion (912) of the rigid portion (910). As a result, the ports (914) in the annular portion (912) of the rigid portion (910) are opened. Similarly, in the open configuration, the radially inward circumferential surface (962) of the annular flap (958) is moved distally out of the groove (918) of the distally extending portion (916) of the rigid portion (910). As a result, the central port (964) in the annular flap (958) of the elastic portion (950) is opened. As such, a fluid flow path (1300) from the proximal drug chamber (40) to the distal drug chamber (42) is opened in the fenestrated separator (900) in the open configuration.

FIGS. 12, 13, and 14 also show that, in some embodiments, a distal end radial expansion (920) at the distal end of the distally extending portion (916). The distal end radial expansion (920) increases the mechanical interference between the elastic portion (950) and the rigid portion (910) to increase the pressure differential between the proximal and distal chambers (40, 42) required to transform the fenestrated separator (900) from the closed configuration to the open configuration. In some embodiments, the distal end radial expansion (920) prevents the seal between the elastic portion (950) and the rigid portion (910) from being inadvertently opened during shipping, handling, or transport. For instance, in some embodiments, ambient air pressure changes may inadvertently transform the fenestrated separator from the closed configuration to the open configuration. Some embodiments are made without a distal end radial expansion at the distal end of the distally extending portion (see e.g., FIG. 11).

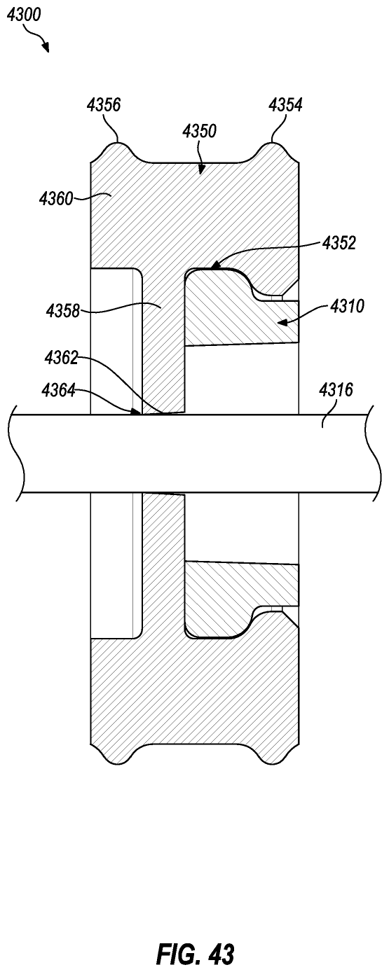

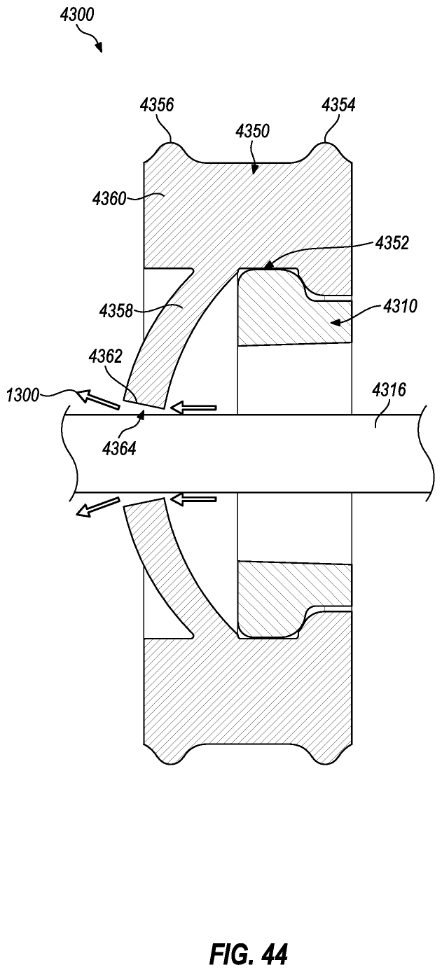

FIGS. 43 and 44 depict a fenestrated separator (4300) for use with multiple chamber serial injection or mix and inject systems, according to various embodiments. The fenestrated separator (4300) is configured to be disposed in the injection system body (34) to define the proximal and distal drug chambers (40, 42) as described above. The fenestrated separator (4300) has a closed configuration (see FIG. 43), in which fluid flow through the fenestrated separator (4300) is prevented, and an open configuration (see FIG. 44), in which fluid is allowed to flow from a proximal chamber to a distal chamber (right to left in FIGS. 43 and 44) through/across the fenestrated separator (4300). The fenestrated separator (4300) is configured to change from the closed configuration to the open configuration with increased pressure in the proximal chamber (the right side of FIGS. 43 and 44) relative to the distal chamber (the left side of FIGS. 43 and 44).

The fenestrated separator (4300) includes a rigid portion (4310) disposed within an elastic portion (4350). The elastic portion (4350) defines a pocket (4352) in which the rigid portion (4310) is disposed. The rigid portion (4310) may be made from a polymer such as cyclic olefin copolymer (COC) and/or a cyclic olefin polymer (COP). Alternatively, the rigid portion (4310) may be made of glass, ceramic, metal, or other non-reactive material with the drug product in the distal drug chamber (42). The elastic portion (4350) may be made from rubber, thermoplastic elastomer (TPE), butyl rubber such as chlorobutyl or bromobutyl, silicone rubber, and/or polyisoprene elastomer.

The fenestrated separator (4300) is also configured to move longitudinally within the injection system body (34; see e.g., FIGS. 6 and 8). The elastic portion (4350) of the fenestrated separator (4300) includes a proximal gasket (4354) and a distal gasket (4356). The proximal and distal gaskets (4354, 4356) are annular bodies that extend radially outward from respective proximal and distal ends of the elastic portion (4350) of the fenestrated separator (4300). The proximal and distal gaskets (4354, 4356) are made from elastic materials and are configured to form first and second fluid-tight seals with an inner surface of the injection system body (34). The elastic materials may be rubber, thermoplastic elastomer (TPE), butyl rubber, silicone rubber, or polyisoprene elastomer. The fenestrated separator (4300) may be PTFE coated on the surfaces facing one or both of the proximal and distal drug chambers (40, 42; see e.g., FIGS. 6 and 8).

While the fenestrated separator (4300) moves longitudinally within the injection system body (34), the fenestrated separator (4300) is configured to maintain contact between the inner surface of the injection system body (34) and both of the proximal and distal gaskets (4354, 4356). To that end, the proximal and distal gaskets (4354, 4356) are separated by a minimum longitudinal distance. Maintaining contact between the inner surface of the injection system body (34) and both of the proximal and distal gaskets (4354, 4356) prevents the fenestrated separator (4300) from twisting or flipping about a diameter thereof, thereby maintaining the respective first and second fluid-tight seals.

The elastic portion (4350) of the fenestrated separator (4300) defines an annular flap (4358) supported by a ring (4360). The annular flap (4358) of the elastic portion (4350) has a radially inward circumferential surface (4362) that defines a central port (4364) through the annular flap (4358). The fenestrated separator (4300) may be disposed around an elongate member (4316). The elongate member (4316) may be a needle of an injection system, which may be retractable to render the injection system safe after injection. The rigid portion (4310) of the fenestrated separator (4300) may define a proximally facing funnel (not shown) to guide the elongate member (4316) to facilitate assembly.

The annular flap (4358) defines a radially inward circumferential surface (4362). When the rigid portion (4310) is disposed in the pocket (4352) in the elastic portion (4350), the radially inward circumferential surface (4362) of the annular flap (4358) of the elastic portion (4350) is biased to form a fluid-tight seal around an outer surface of the elongate member (4316) to fluidly seal/close the central port (4364) in the annular flap (4358) of the elastic portion (4350).

FIG. 43 is a side cross-sectional view of a fenestrated separator (4300) in a closed configuration, according to some embodiments. In the closed configuration, the radially inward circumferential surface (4362) of the annular flap (4358) of the elastic portion (4350) is relatively aligned with the longitudinal axis of the elongate member (4316) and fluidly seals/closes around the outer surface of the elongate member (4316). As such, fluid flow from the proximal chamber (the right side of FIGS. 43 and 44) to the distal chamber (the left side of FIGS. 43 and 44) is prevented by the fenestrated separator (4300) in the closed configuration.

FIG. 44 is a side cross-sectional view of a fenestrated separator (4300) in an open configuration, according to some embodiments. In the open configuration, increased pressure in the proximal chamber (the right side of FIGS. 43 and 44) relative to the distal chamber (the left side of FIGS. 43 and 44) deforms/domes the annular flap (4358) of the elastic portion (4350) distally away from the rigid portion (4310). As a result, the radially inward circumferential surface (4362) of the annular flap (4358) of the elastic portion (4350) moves away from and rotates out of alignment with the longitudinal axis of the elongate member (4316). As a result, the central port (4364) in the annular flap (4358) of the elastic portion (4350) is opened. As such, a fluid flow path (1300) from the proximal chamber (the right side of FIGS. 43 and 44) to the distal chamber (the left side of FIGS. 43 and 44) is opened in the fenestrated separator (4300) in the open configuration.

### 2. Fenestrated Separator Operation

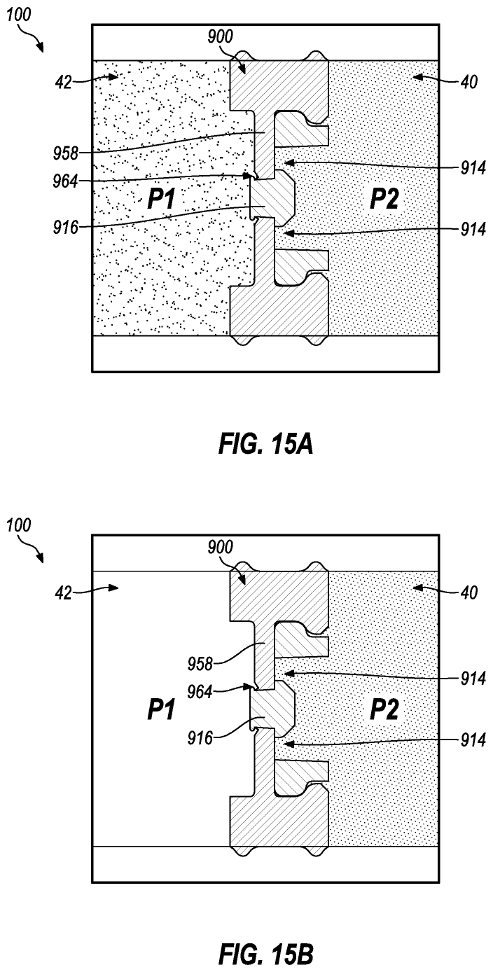

FIGS. 15A to 17B are detailed side cross-sectional views depicting a fenestrated separator (900) opening and closing in a serial injection system (100) to facilitate serial injection, according to some embodiments. The fenestrated separator (900) opens and closes in response to applied force and pressure differentials in the proximal and distal drug chambers (40, 42). In FIG. 15A, the first and second liquids (252, 254) are disposed in distal and proximal drug chambers (42, 40), respectively. Consequently, the pressure (P1) in the distal drug chamber (42) and the pressure (P2) in the proximal drug chamber (40) are equal. In this step in the serial injection method, the bias of the annular flap (958) places the fenestrated separator (900) in the closed configuration. In the closed configuration distally directed force applied to the stopper member (32) through the plunger member (44) is transferred through the incompressible second liquid (254) to the fenestrated separator (900), thereby moving the fenestrated separator (900) distally along a longitudinal axis of the injection system body (34) and ejecting the first liquid (252) from the distal drug chamber (42) as shown in FIG. 15B.

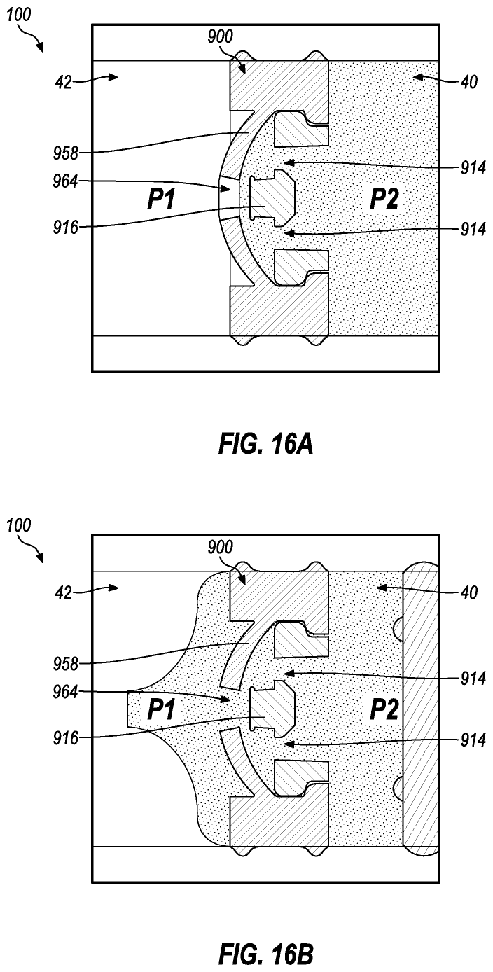

In FIG. 15B, the first liquid (252) has been mostly ejected from the distal drug chamber (42) by distal movement of the fenestrated separator (900). As such, the pressure (P2) in the proximal drug chamber (40) increases relative to the pressure (P1) in the distal drug chamber (42). As a result, the fenestrated separator (900) begins to transform from the closed configuration to the open configuration as shown in FIG. 16A.

In FIG. 16A, continued application of distally directed force to the stopper member (32) through the plunger member (44) increases the pressure (P2) in the proximal drug chamber (40) relative to the pressure (P1) in the distal drug chamber (42). The increased pressure in the proximal drug chamber (40) relative to the distal drug chamber (42) deforms/domes the annular flap (958) of the elastic portion (950) distally away from the annular portion (912) of the rigid portion (910) to transform the fenestrated separator (900) from the closed configuration to the open configuration. With the fenestrated separator (900) in the open configuration, the second liquid (254) can flow from the proximal drug chamber (40) to the distal drug chamber (42) through the fenestrated separator (900) as shown in FIG. 16B.

In FIG. 16B, some of the second liquid (254) has flowed from the proximal drug chamber (40) to the distal drug chamber (42), but the pressure (P2) in the proximal drug chamber (40) remains higher relative to the pressure (P1) in the distal drug chamber (42). As such, the fenestrated separator (900) remains in the open configuration.

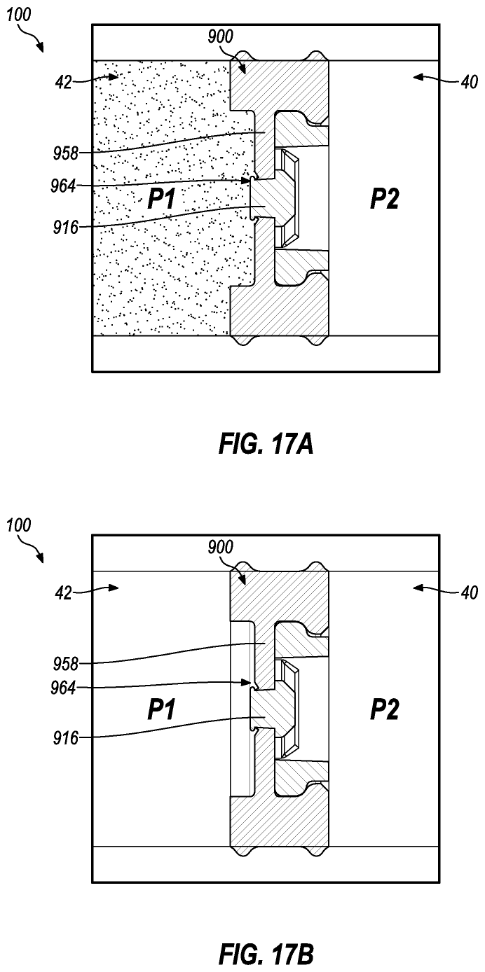

In FIG. 17A, most of the second liquid (254) has flowed from the proximal drug chamber (40) to the distal drug chamber (42). Consequently, the pressure (P1) in the distal drug chamber (42) rises and the pressure (P2) in the proximal drug chamber (40) drops, until the pressure (P2) in the proximal drug chamber (40) is lower relative to the pressure (P1) in the distal drug chamber (42). As a result, the fenestrated separator (900) transforms from the open configuration to the closed configuration. In the closed configuration, distally directed force applied to the stopper member (32) through the plunger member (44) is transferred through the incompressible second liquid (254) to the fenestrated separator (900), thereby moving the fenestrated separator (900) distally along a longitudinal axis of the injection system body (34) and ejecting the second liquid (254) from the distal drug chamber (42) as shown in FIG. 17B.

In FIG. 17B, most of the second liquid (254) has been ejected from the distal drug chamber (42). Consequently, the pressure (P1) in the distal drug chamber (42) and the pressure (P2) in the proximal drug chamber (40) are equal. In this step in the serial injection method, the bias of the annular flap (958) maintains the fenestrated separator (900) in the closed configuration.

### 3. Exemplary Serial Injection Method

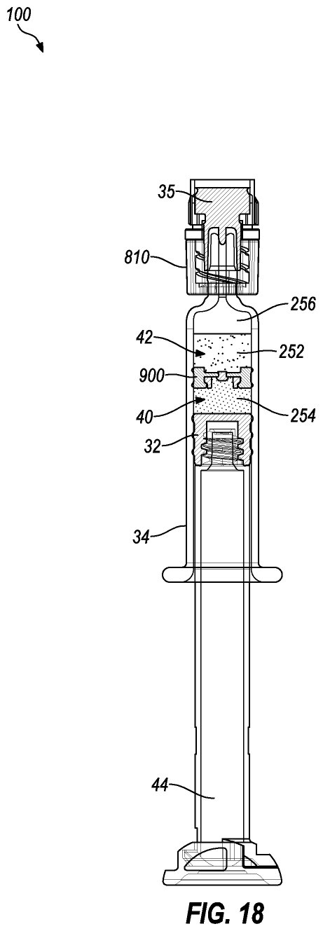

FIGS. 18 to 29 are side cross-sectional views depicting various steps in a serial injection method using a dual chamber serial injection system (100), according to some embodiments. FIG. 18 depicts a prefilled dual chamber serial injection system (100) in a transport/storage configuration. As described herein, the dual chamber serial injection system (100) includes a conventional off-the-shelf injection system body (34), a conventional off-the-shelf stopper member (32) disposed therein, a fenestrated separator (900), proximal and distal drug chambers (40, 42), a plunger member (44), a distal needle interface (810) and a distal seal or cap (35) closing the distal needle interface (810). First and second liquids (252, 254) are contained in the distal and proximal drug chambers (42, 40), respectively. An air bubble (256) is also contained in the distal drug chamber (42) to facilitate mixing of the first liquid (252), which may be a drug suspension (e.g., an aluminum suspension).

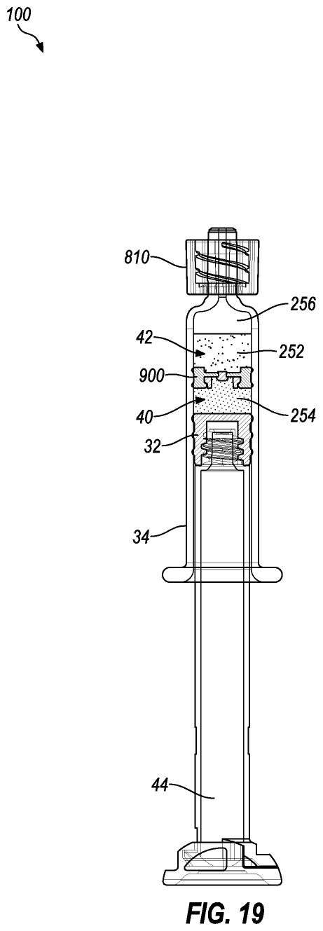

In the step depicted in FIG. 19, the dual chamber serial injection system (100) is shaken to mix/re-suspend the first liquid (252). Also, the distal sealer (35) is removed from the distal needle interface (810) to prepare the system (100) for injection.

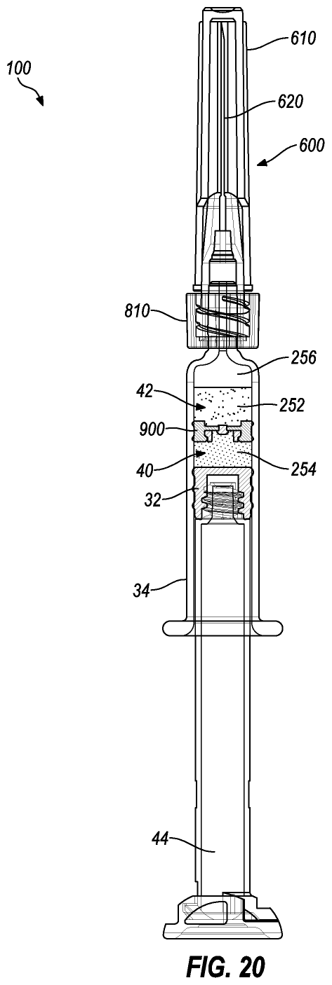

In the step depicted in FIG. 20, a needle assembly (600) is coupled to the distal needle interface (810) to prepare the system (100) for injection.

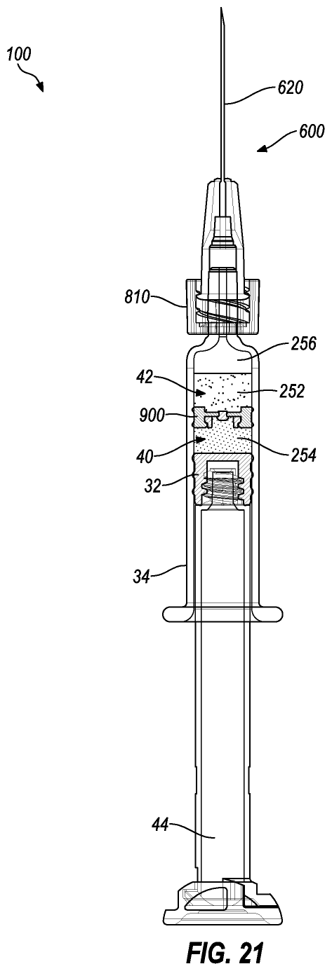

In the step depicted in FIG. 21, a needle shield (610) is removed from the needle assembly (600) to expose the needle (620) to prepare the system (100) for injection.

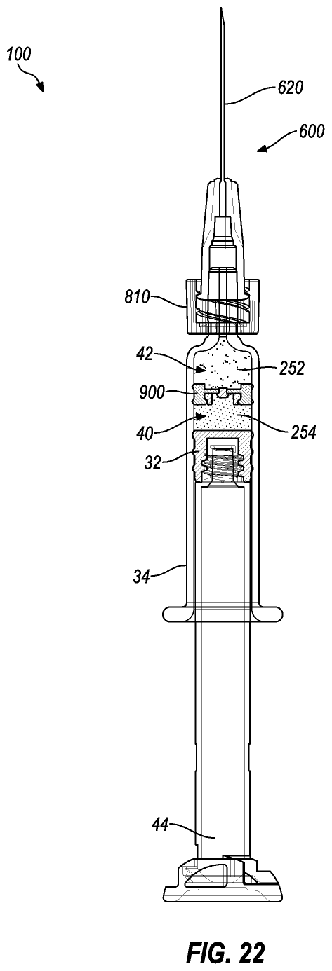

In the step depicted in FIG. 22, the dual chamber serial injection system (100) is position with the needle pointing upwards to move the air bubble (256) toward the distal needle interface (810). In the steps depicted in FIGS. 18 to 24, the fenestrated separator (900) is in a closed configuration shown in FIG. 15A. Then a distally directed force is applied to the plunger member (44) and the stopper member (32) attached thereto. The distally directed force is transferred through the incompressible second liquid (254) to the fenestrated separator (900). With the fenestrated separator (900) in the closed configuration, the distally directed force transferred to the fenestrated separator (900) moves the fenestrated separator (900) distally along a longitudinal axis of the injection system body (34) and ejecting the air bubble (256) from the distal drug chamber (42) to “de-bubble” the system (100). After the system (100) is de-bubbled, the system (100) is ready for injection.

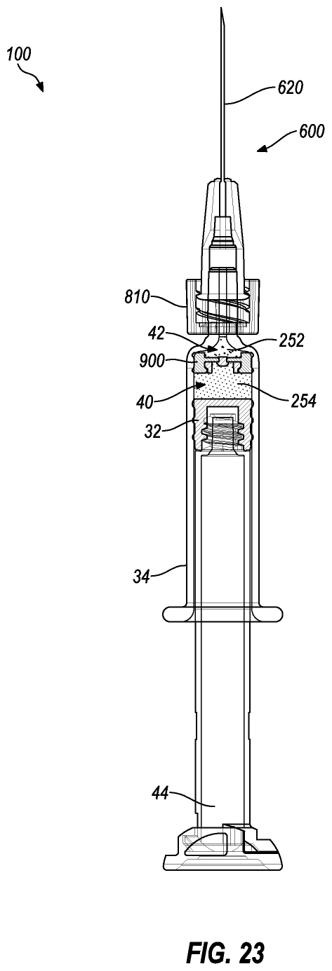

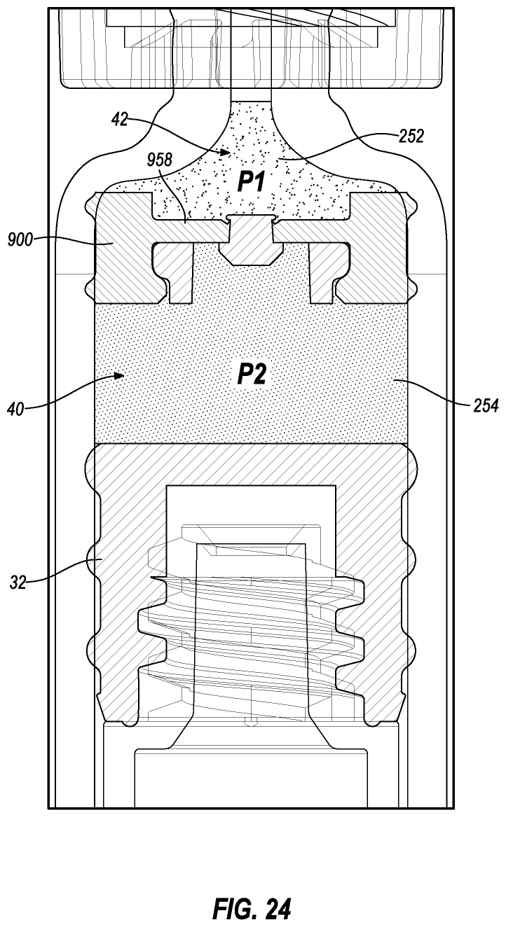

In the step depicted in FIGS. 23 and 24, the sharp distal end (622) of the needle (620) may be inserted into an injection target (e.g., a patient). Then the distally directed force is further applied to the plunger member (44) and the stopper member (32) attached thereto. The distally directed force is transferred through the incompressible second liquid (254) to the fenestrated separator (900). With the fenestrated separator (900) in the closed configuration, the distally directed force transferred to the fenestrated separator (900) moves the fenestrated separator (900) distally along a longitudinal axis of the injection system body (34) and ejecting some of the first liquid (252) from the distal drug chamber (42).

FIG. 24 is a detailed side cross-sectional view depicting the configuration of the fenestrated separator (900) in the serial injection step depicted in FIG. 23. During the step depicted in FIGS. 23 and 24, the pressure (P1) in the distal drug chamber (42) and the pressure (P2) in the proximal drug chamber (40) are equal, and the bias of the annular flap (958) places the fenestrated separator (900) in the closed configuration as shown in FIG. 15A and described above.

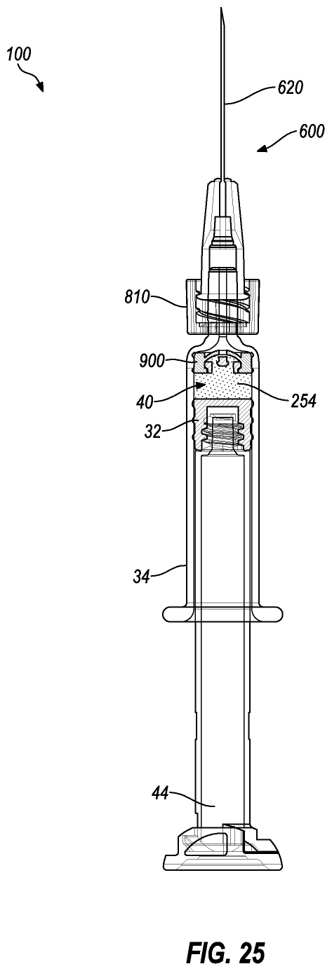

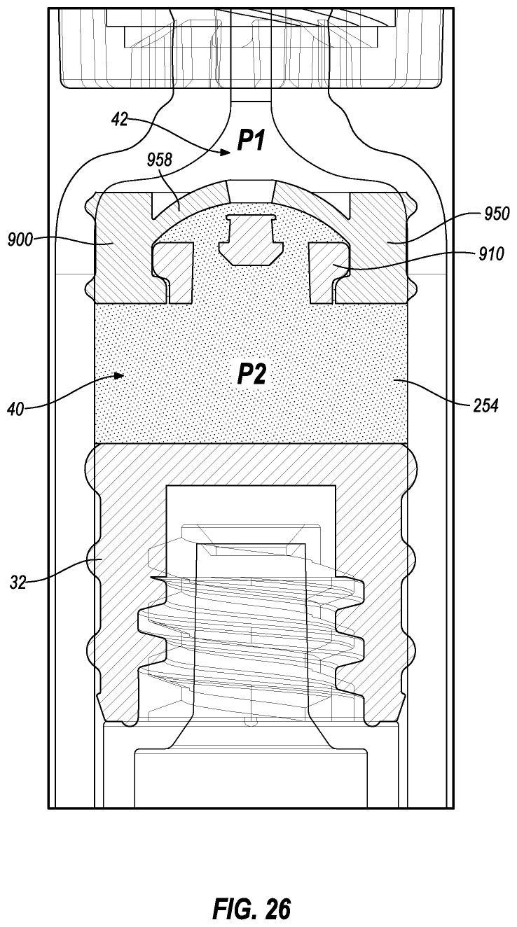

In the step depicted in FIGS. 25 and 26, the distally directed force is still further applied to the plunger member (44) and the stopper member (32) attached thereto. Because the first liquid (252) has been mostly ejected from the distal drug chamber (42) by distal movement of the fenestrated separator (900), the pressure (P2) in the proximal drug chamber (40) increases relative to the pressure (P1) in the distal drug chamber (42). As a result, the fenestrated separator (900) begins to transform from the closed configuration to the open configuration as shown in FIG. 16A and described above.

FIG. 26 is a detailed side cross-sectional view depicting the configuration of the fenestrated separator (900) in the serial injection step depicted in FIG. 25. During the step depicted in FIGS. 25 and 26, the increased pressure in the proximal drug chamber (40) relative to the distal drug chamber (42) deforms/domes the annular flap (958) of the elastic portion (950) distally away from the annular portion (912) of the rigid portion (910) to transform the fenestrated separator (900) from the closed configuration to the open configuration. With the fenestrated separator (900) in the open configuration, the second liquid (254) can flow from the proximal drug chamber (40) to the distal drug chamber (42) through the fenestrated separator (900) as shown in FIGS. 16B, 27, and 28, and described above.

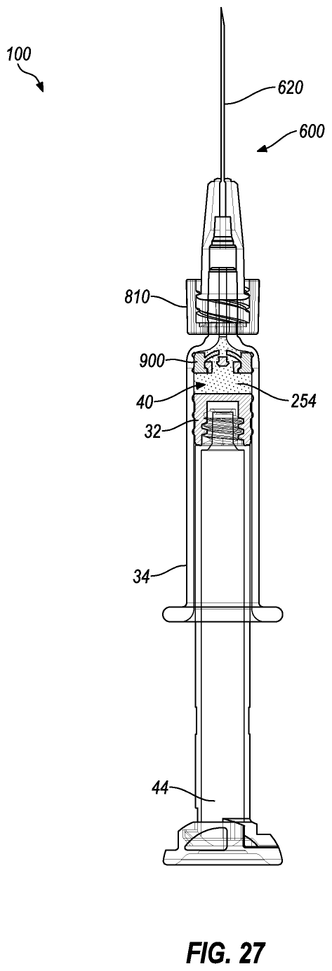

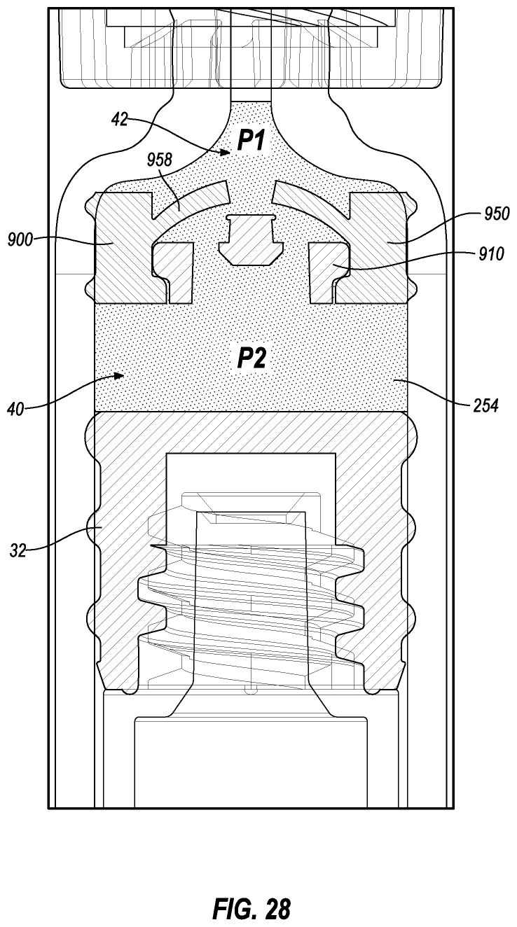

In the step depicted in FIGS. 27 and 28, the distally directed force is even further applied to the plunger member (44) and the stopper member (32) attached thereto. While some of the second liquid (254) has flowed from the proximal drug chamber (40) to the distal drug chamber (42), the pressure (P2) in the proximal drug chamber (40) remains higher relative to the pressure (P1) in the distal drug chamber (42). As such, the fenestrated separator (900) remains in the open configuration. as shown in FIG. 16B and described above. Continued application of the distally directed force to the plunger member (44) in the stopper member (32) attached thereto ejects the second liquid (254) from the distal drug chamber (42) and out the needle (620), as shown in FIGS. 17A and 17B and described above.

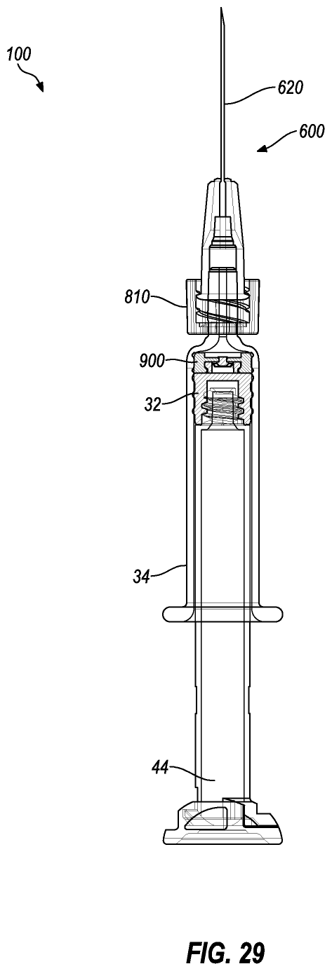

In the step depicted in FIG. 29, most of the second liquid (254) has been ejected from the distal drug chamber (42). Consequently, the pressure (P1) in the distal drug chamber (42) rises and the pressure (P2) in the proximal drug chamber (40) drops, until the pressure (P2) in the proximal drug chamber (40) is lower relative to the pressure (P1) in the distal drug chamber (42). As a result, the fenestrated separator (900) transforms from the open configuration to the closed configuration. as shown in FIG. 17B and described above.

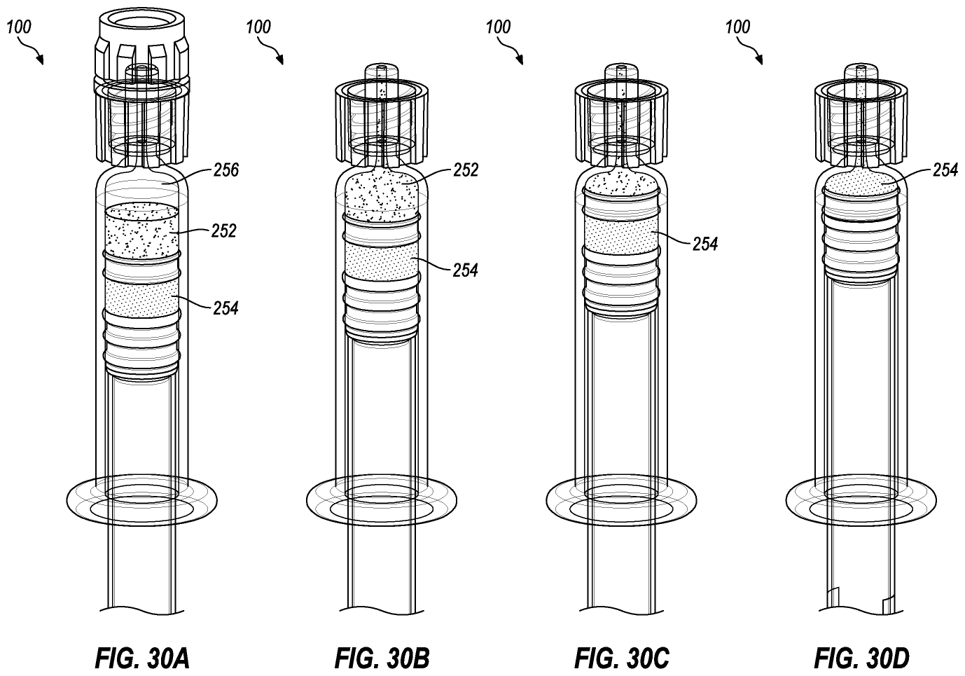

FIGS. 30A to 30D are schematically depict various steps in a serial injection method using a dual chamber serial injection system (100), according to some embodiments. FIG. 30A depicts the system (100) in a transport/storage configuration as shown in FIG. 18 and described above. FIG. 30B depicts the system (100) after de-bubbling as shown in FIG. 22 and described above. FIG. 30C depicts the system (100) after the first liquid (252), which is blue in this embodiment, is almost fully ejected from the system (100). FIG. 30D depicts the system (100) after the second liquid (254), which is green in this embodiment, is almost fully ejected from the system (100).

As shown in the prefilled dual chamber serial injection systems (100), various components of the systems (100) and their positions can be modified to tailor the amounts of first and second liquids (252, 254) to be injected, including portions of the first liquid (252) that may be injected before and after the second liquid (254).

The amount of force need to transform the fenestrated separator (900) from the closed configuration to the open configuration can be modulated to accommodate a combination of the system function requirements and the aesthetic impression on the user. If the activation force is too low, it may work, but be too difficult for the user to apply the force lightly enough, and the fenestrated separator (900) may open too early. If the force is too high, the user may find that it is “too hard” to open the fenestrated separator (900). Fortunately, the predetermined amount of force can be “tune” a range by modifying various component characteristics, including but not limited to, one or more of the sizes of the radially inward circumferential surface (962) of the annular flap (958), the groove (918) of the distally extending portion (916), and the distal end radial expansion (920) at the distal end of the distally extending portion (916).

### Exemplary Triple Chamber Liquid Mixing and Injection System and Method

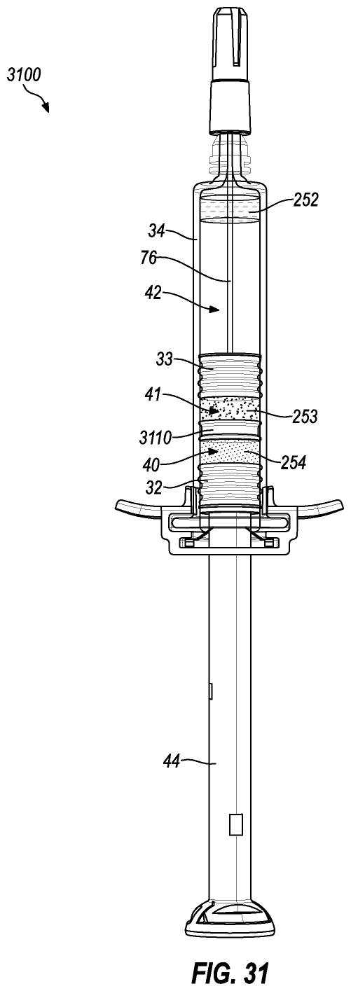

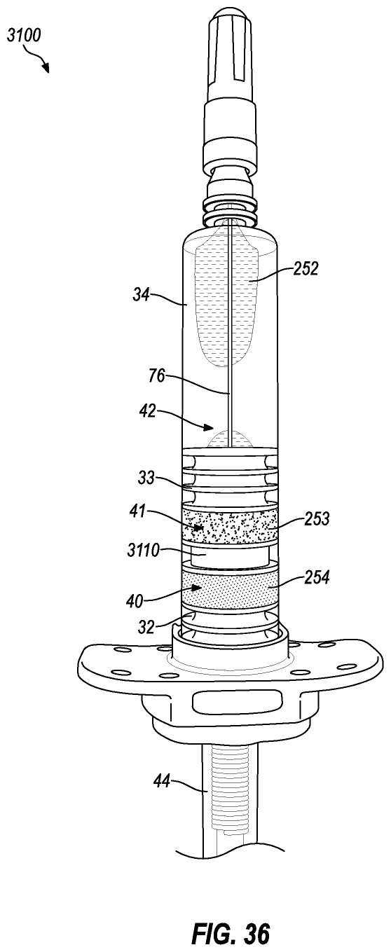

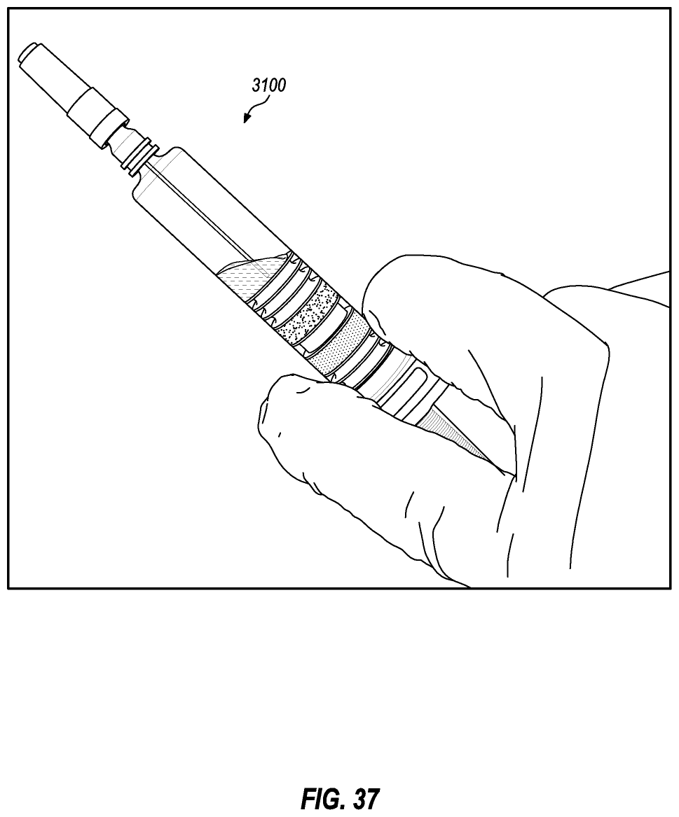

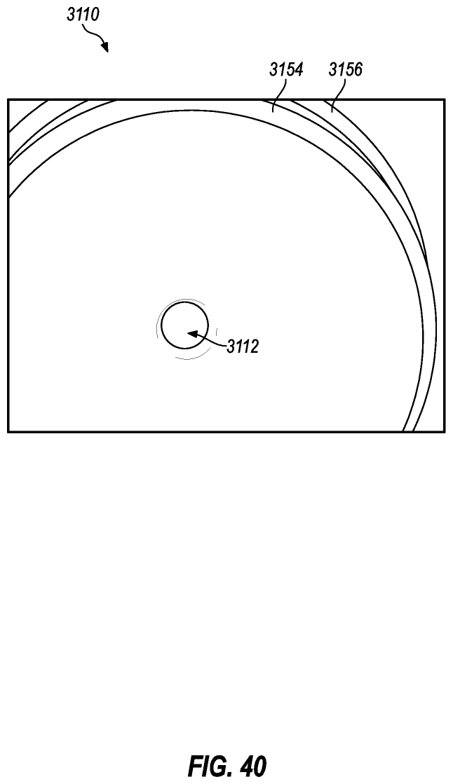

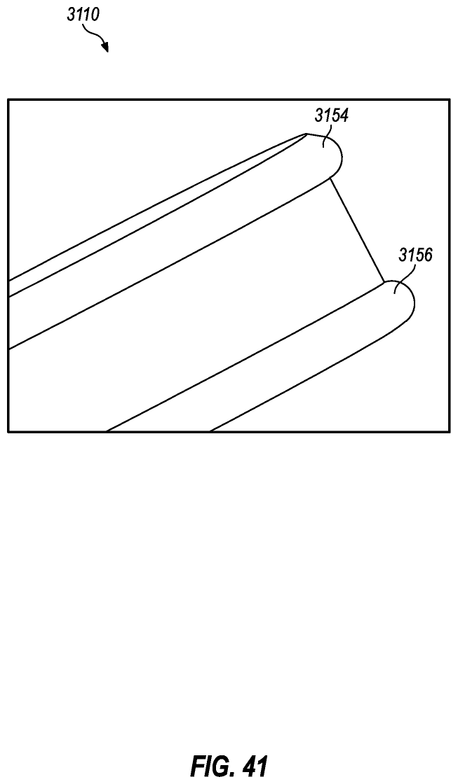

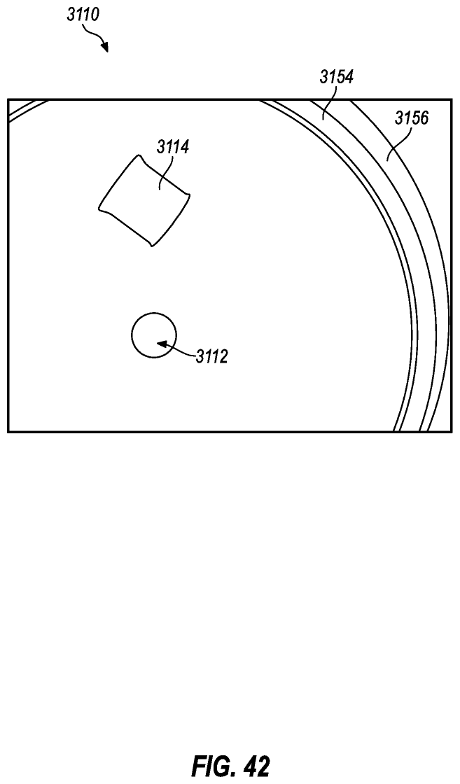

FIGS. 31 to 42 depict a triple chamber liquid mixing and injection system (3100), according to some embodiments. The triple chamber liquid mixing and injection system (3100) is configured to mixed three liquids (e.g., drug components) before injecting the mixed liquid (e.g., drug).

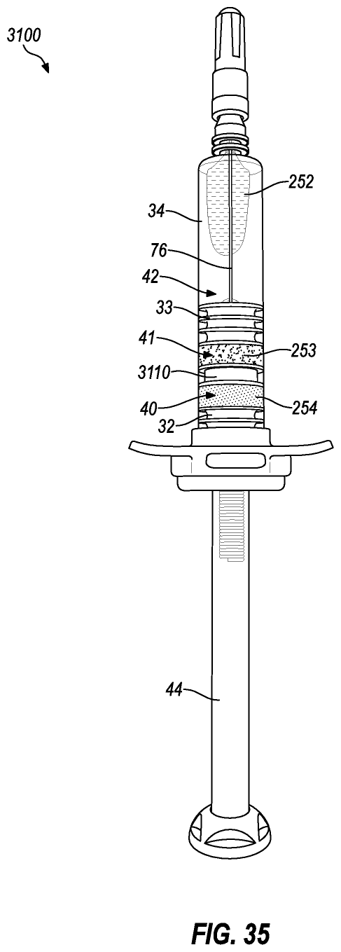

FIG. 31 is a side view of a triple chamber liquid mixing and injection system (3100) that is prefilled with first, second, and third liquids (252, 253, 254), which may be first, second, and third drug components for mixing before injection, according to some embodiments. The triple chamber liquid mixing and injection system (3100) includes a conventional off-the-shelf injection system body (34) with a conventional off-the-shelf proximal stopper member (32) disposed therein. The triple chamber liquid mixing and injection system (3100) also includes a middle fenestrated separator (3110) disposed in the injection system body (34) at a first distance distal of the stopper member (32). The triple chamber liquid mixing and injection system (3100) further includes a distal stopper member (33) disposed in the injection system body (34) at a second distance distal of the middle fenestrated separator (3110). The proximal stopper member (32) and the middle fenestrated separator (3110) together with the injection system body (34) define a proximal drug chamber (40). The middle fenestrated separator (3110) and the distal stopper member (33) together with the injection system body (34) define a middle drug chamber (41). The distal stopper member (33) together with the injection system body (34) define a distal drug chamber (42). First, second, and third liquids (252, 253, 254) are contained in the distal, middle, and proximal drug chambers (42, 41, 40), respectively (see e.g., FIGS. 31 and 35). The proximal stopper member (32) and the middle fenestrated separator (3110) occlude the proximal and distal ends of the proximal drug chamber (40), respectively. The middle fenestrated separator (3110) and the distal stopper member (31) occlude the proximal and distal ends of the middle drug chamber (41), respectively. The distal stopper member (31) occludes the proximal end of the distal drug chamber (42).

The triple chamber liquid mixing and injection system (3100) also includes a needle (76) with a needle proximal end (50). The triple chamber liquid mixing and injection system (3100) further includes a plunger member (44) coupled to the proximal stopper member (32) and configured to insert the proximal stopper member (32) distally into the injection system body (34).

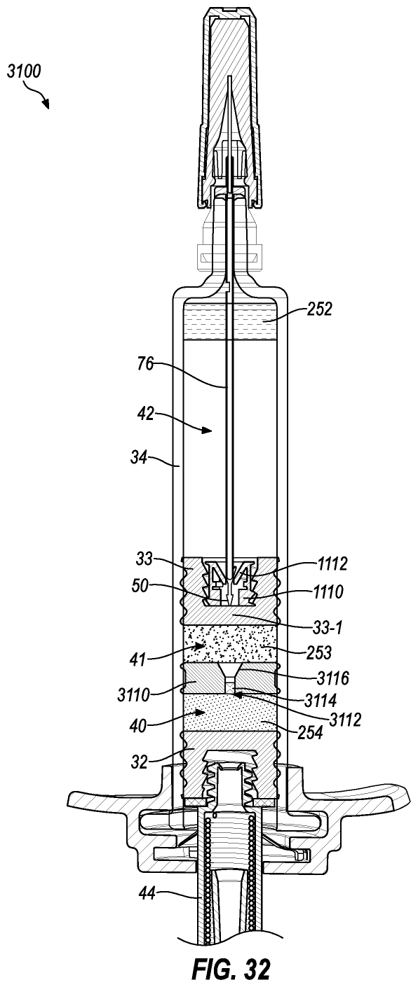

The proximal stopper member (32) may be a conventional off-the-shelf stopper member according to some embodiments. The proximal stopper member (32) may be coupled to the plunger member (44) with a screw-type connector as shown in FIGS. 32 to 34.

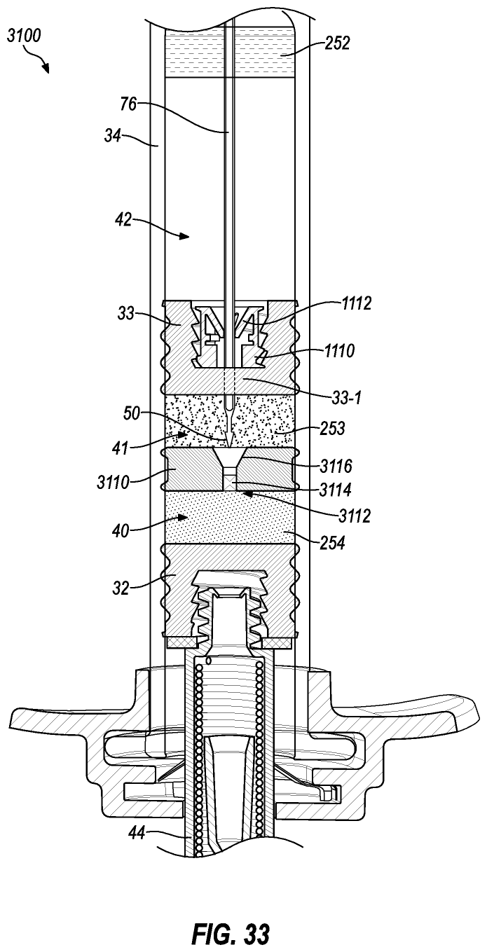

The middle fenestrated separator (3110) is a fenestrated separator (3110) for use with dual chamber injection systems (3100), according to some embodiments. The middle fenestrated separator (3110) is configured to be disposed in the injection system body (34) to define the proximal and middle drug chambers (40, 41) as described above. The middle fenestrated separator (3110) defines a middle port (3112). The middle fenestrated separator (3110) also includes a plug (3114) removably disposed in the middle port (3112). The middle fenestrated separator (3110) also defines a distally facing funnel (3116) configured to guide the needle proximal end (50) toward the middle port (3112), as shown in FIG. 33, to dislodge the plug (3114) therefrom with distal movement of the middle fenestrated separator (3110) relative to the needle (76) and needle proximal end (50).

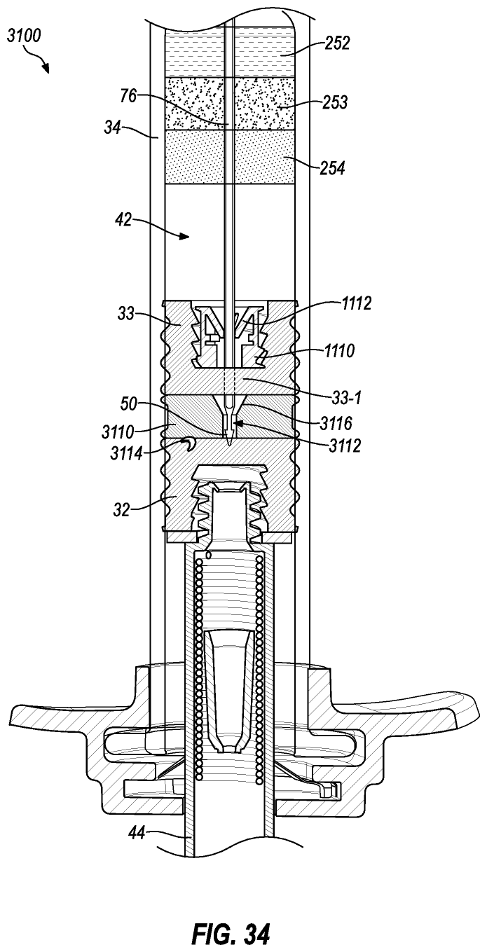

The middle fenestrated separator (3110) has a closed configuration (see FIGS. 32 and 33), in which the plug (3114) is disposed in the middle port (3112) to prevent fluid flow through the middle fenestrated separator (3110). The middle fenestrated separator (3110) also has an open configuration (see FIG. 34), in which the plug (3114) is dislodged from the middle port (3112) to allow fluid to flow from the proximal drug chamber (40) to the middle drug chamber (41) and to the distal drug chamber (42) through the middle fenestrated separator (3110). The middle fenestrated separator (3110) is configured to change from the closed configuration to the open configuration with distal movement of the middle fenestrated separator (3110) relative to the needle (76) and needle proximal end (50) to dislodge the plug (3114) with the middle port (3112). After the plug (3114) is dislodged from the middle port (3112), it is disposed in the proximal drug chamber (40), and after the third liquid (254) is transferred from the proximal drug chamber (40) to the middle drug chamber (41), the plug (3114) is disposed between and in contact with both the middle fenestrated separator (3110) and the proximal stopper member (32), which are also in contact with each other.

The plug (3114) and the middle port (3112) may be tuned as described in U.S. Utility patent application Ser. No. 18/377,601, which was previously incorporated by reference herein, to control the amount of proximally directed force required to dislodge the plug (3114) with the middle port (3112).

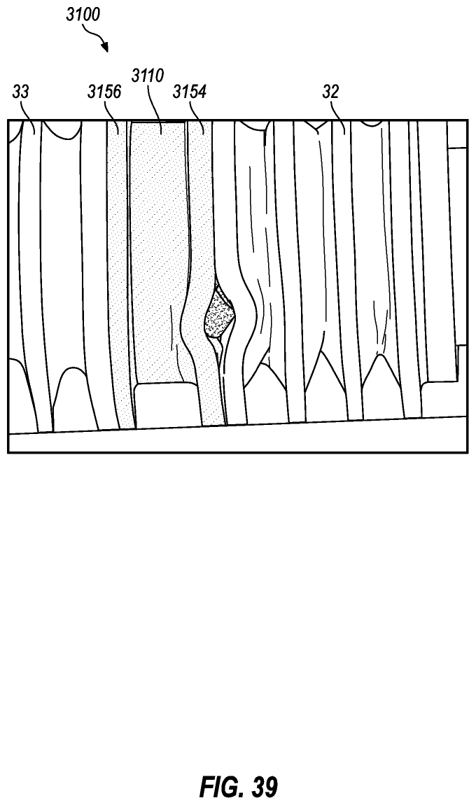

The middle fenestrated separator (3110) includes a proximal gasket (3154) and a distal gasket (3156), as shown in FIGS. 39 to 42. The proximal and distal gaskets (3154, 3156) are annular bodies that extend radially outward from respective proximal and distal ends of the middle fenestrated separator (3110). The proximal and distal gaskets (3154, 3156) are made from elastic materials and are configured to form first and second fluid-tight seals with an inner surface of the injection system body (34). The elastic materials may be rubber, thermoplastic elastomer (TPE), butyl rubber, silicone rubber, or polyisoprene elastomer. The middle fenestrated separator (3110) may be PTFE coated on the surfaces facing one or both of the proximal and middle drug chambers (40, 41).

While the middle fenestrated separator (3110) moves longitudinally within the injection system body (34), the middle fenestrated separator (3110) is configured to maintain contact between the inner surface of the injection system body (34) and both of the proximal and distal gaskets (3154, 3156). To that end, the proximal and distal gaskets (3154, 3156) are separated by a minimum longitudinal distance. Maintaining contact between the inner surface of the injection system body (34) and both of the proximal and distal gaskets (3154, 3156) prevents the middle fenestrated separator (3110) from twisting or flipping about a diameter thereof, thereby maintaining the respective first and second fluid-tight seals.

The distal stopper member (33) may be a conventional off-the-shelf stopper member according to some embodiments. As shown in FIG. 32, the direction of the conventional distal stopper member (33) is opposite of the typical direction with the uninterrupted surface, which typically faces distally, facing proximally. The distal stopper member (33) may include a stopper bushing (1110) defining a distally directed funnel (1112) configured to guide the needle proximal end (50) to a central portion (33-1) of the distal stopper member (33). Further details regarding the distal stopper member (33) are described in U.S. Utility patent application Ser. No. 16/435,429, which was previously incorporated by reference herein.

The distal stopper member (33) has a closed configuration (see FIG. 32), in which the central portion (33-1) of the distal stopper member (33) is whole/continuous/unbroken and forms a barrier to prevent fluid flow through the distal stopper member (33). The distal stopper member (33) also has an open configuration (see FIGS. 33 and 34), in which the central portion (33-1) of the distal stopper member (33) is pierced by the needle proximal end (50) to allow fluid to flow from the middle drug chamber (41) to the distal drug chamber (42) through the distal stopper member (33). The distal stopper member (33) is configured to change from the closed configuration to the open configuration with distal movement of the distal stopper member (33) relative to the needle (76) and needle proximal end (50) to pierce the central portion (33-1) of the distal stopper member (33).

In some embodiments, a distal surface of the proximal stopper member (32), proximal and distal surfaces of the middle fenestrated separator (3110), and/or a proximal surface of the distal stopper member (33) are coated with a lubricious polymer coating (e.g., PTFE or ETFE), the polymer coatings on these surfaces, together with the injection system body (34) define the proximal, middle, and distal drug chambers (40, 41, 42). The lubricious polymer coating also serves to isolate the rubber of the proximal stopper member (32), the middle fenestrated separator (3110), and the distal stopper member (33) from the first, second, and third liquids (252, 253, 254). In other embodiments, the proximal stopper member (32), the middle fenestrated separator (3110), and/or the distal stopper member (33) may be constructed from un-coated butyl rubber such as chlorobutyl and/or bromobutyl rubber. Alternatively, the proximal stopper member (32), the middle fenestrated separator (3110), and/or the distal stopper member (33) may be constructed of other elastomeric materials such as TPE, TPU, silicone rubber, or other elastomers.

The triple chamber liquid mixing and injection system (3100) facilitates mixing of the first, second, and third liquids (252, 253, 254) from the distal drug chamber (42), the middle drug chamber (41), and the proximal drug chamber (40), respectively, in the distal drug chamber (42) before injection in a short time after mixing. In this way, the triple chamber liquid mixing and injection system (3100) facilitates injection of drugs including three components that degrade rapidly after mixing of the three components.

FIGS. 31, 32, and 35 to 37 depict the triple chamber liquid mixing and injection system (3100) in a storage/transport configuration in which the first, second, and third liquids (252, 253, 254) are stored in the distal drug chamber (42), the middle drug chamber (41), and the proximal drug chamber (40), respectively. In the storage/transport configuration, the distal stopper member (33) and the middle fenestrated separator (3110) are in their respective closed configurations as described herein. As such, the first, second, and third liquids (252, 253, 254) are isolated from each other to prevent degradation from mixing the liquids.

FIG. 33 depicts the triple chamber liquid mixing and injection system (3100) in a first opened configuration, in which distal movement of the distal stopper member (33) relative to the needle (76) and needle proximal end (50) causes the needle proximal end (50) to pierce the central portion (33-1) of the distal stopper member (33) to convert the distal stopper member (33) from the close to the open configuration as described herein. The triple chamber liquid mixing and injection system (3100) can be configured such that a distally directed force applied to the plunger member (44) is transferred through the proximal stopper member (32), through the third liquid (254) (via incompressibility of same), through the middle fenestrated separator (3110), through the second liquid (253) (via incompressibility of same), to the distal stopper member (33) to cause the distal movement of the distal stopper member (33) relative to the needle (76) and needle proximal end (50). The distal stopper member (33) being in the open configuration allows the second liquid (253) to flow from the middle drug chamber (41) through the distal stopper member (33) along a reduced diameter section of the needle (76) to the distal drug chamber (42) and to mix with the first liquid (252) contained in the distal drug chamber (42). The first, second, and third liquids (252, 253, 254) may be selected such that mixing the first and second liquids (252, 253) results in less degradations than mixing the third liquid (254) with the first and second liquids (252, 253) in order to minimize degradation before injection of the mixed liquid.

FIG. 34 depicts the triple chamber liquid mixing and injection system (3100) in a second opened configuration, in which further distal movement of the distal stopper member (33) relative to the needle (76) and needle proximal end (50) causes the needle proximal end (50) to dislodge the plug (3114) from the middle port (3112) in the middle fenestrated separator (3110) to convert the middle fenestrated separator (3110) from the close to the open configuration as described herein. The middle fenestrated separator (3110) being in the open configuration allows the third liquid (254) to flow from the proximal drug chamber (40) through middle drug chamber (41) and the distal stopper member (33) to the distal drug chamber (42) and to mix with the first and second liquids (252, 253) contained in the distal drug chamber (42).

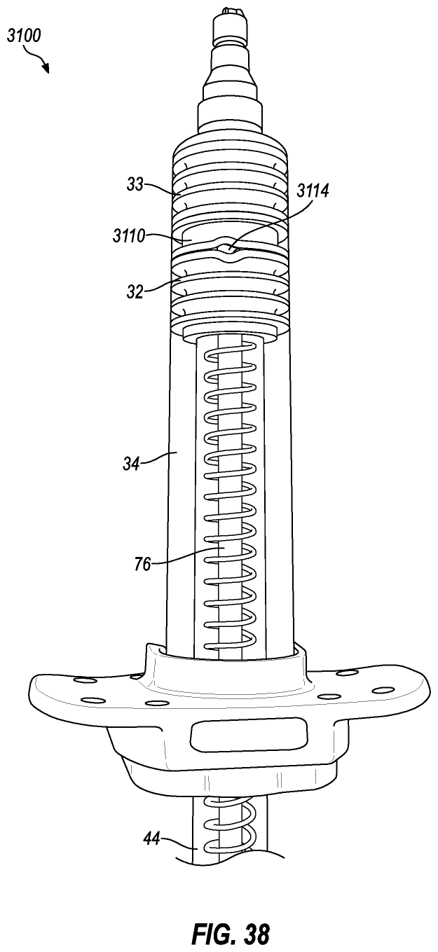

FIG. 38 depicts the triple chamber liquid mixing and injection system (3100) in a post-injection configuration, in which still further distal movement of the distal stopper member (33), the middle fenestrated separator (3110), and the proximal stopper member (32) relative to the needle (76), the needle proximal end (50) and the injection system body (34) ejects substantially all of the mixed liquid formed by mixing the first, second, and third liquids (252, 253, 254) from the triple chamber liquid mixing and injection system (3100). Advancing the distal stopper member (33) adjacent the distal end of the injection system body (34) also releases a spring to retract the needle (76) until a sharp distal end of the needle (76) is disposed at least inside of the needle hub attached to the injection system body (34) or the injection system body (34) to render the triple chamber liquid mixing and injection system (3100) “safe” (i.e., from accidental needle sticks post injection). Further details regarding the needle retraction system are described in U.S. Utility patent application Ser. No. 14/696,342 and U.S. Pat. No. 10,010,677, which were previously incorporated by reference herein. In brief, additional components of the triple chamber liquid mixing and injection system (3100) include a needle retraction system in the interior of the plunger member (44): a needle retention feature; an energy-storage member (e.g., spring); an energy-storage member latch; and a needle holder member (e.g., O-ring, needle latches, and/or detents) in the interior of a needle hub.

While the middle fenestrated separator (3110) in the triple chamber liquid mixing and injection system (3100) described herein is disposed between proximal and distal stopper members (32, 34), other embodiments of triple chamber liquid mixing and injection systems may include two (middle and distal) fenestrated separators with structure and function similar to the middle fenestrated separator (3110). Similarly, other embodiments of dual chambers injection systems may include a distal fenestrated separator with structure and function similar to the middle fenestrated separator (3110) instead of a distal stopper member.

While some of the embodiments described herein do not include a safe needle retraction system, the embodiments can be utilized with multiple chamber safe injection systems with needle retraction, such as those disclosed in U.S. Utility patent application Ser. No. 16/435,429 and 17/364,546, which were previously incorporated by reference herein. In brief, additional components of the dual chamber serial injection system (100) may include a needle retraction system in the interior of the plunger member (44): a needle retention feature; an energy-storage member (e.g., spring); an energy-storage member latch; a needle holder member (e.g., O-ring, needle latches, and/or detents) in the interior of the needle hub; and a funnel to guide a needle proximal end during injection and retraction.

While the embodiments described above include dual chamber and triple chamber injection systems, the scope of the claims also include other multiple chamber injection systems. For multiple chamber injection systems with more than two or three chambers, more than two fenestrated separators or stopper members are inserted into an injection system body (e.g., syringe body, cartridge body, etc.) to define a corresponding number of chambers.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, ETFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. An injection system, comprising:
an injection system body defining a proximal opening at a proximal end thereof and a distal needle interface at a distal end thereof;
a stopper member and a fenestrated separator disposed in the injection system body, forming a proximal drug chamber between the stopper member and the fenestrated separator and a distal drug chamber between the fenestrated separator and the distal end of the injection system body; and
a plunger member configured to be manually manipulated to insert the stopper member relative to the injection system body;
wherein the fenestrated separator forms an openable barrier between the proximal and distal drug chambers, and
wherein the fenestrated separator is configured to allow flow from the proximal drug chamber to the distal drug chamber with increased pressure in the proximal drug chamber relative to the distal drug chamber.

2. The system of claim 1, wherein the fenestrated separator is configured to move longitudinally within the injection system body.

3. The system of claim 1, the fenestrated separator comprising proximal and distal gaskets disposed adjacent respective proximal and distal ends thereof and extending radially therefrom.

4. The system of claim 3, wherein the fenestrated separator is configured to maintain contact between an inner surface of the injection system body and the proximal gasket and between the inner surface of the injection system body and the distal gasket as the fenestrated separator moves longitudinally within the injection system body.

5. The system of claim 4, wherein the proximal and distal gaskets are separated by a minimum longitudinal distance.

6. The system of claim 3, wherein the proximal and distal circumferential gaskets are configured to form respective first and second fluid-tight seals with an inner surface of the injection system body.

7. The system of claim 3, wherein the proximal and distal circumferential gaskets are made from an elastic material.

8. The system of claim 7, wherein the elastic material is rubber, thermoplastic elastomer, butyl rubber, or polyisoprene elastomer.

9. The system of claim 1, wherein the fenestrated separator comprises a port configured to be opened by the increased pressure in the proximal drug chamber relative to the distal drug chamber.

10. The system of claim 1, wherein the fenestrated separator comprises a rigid portion and an elastic portion.

11. The system of claim 10, wherein the elastic portion defines a pocket configured to receive the rigid portion therein.

12. The system of claim 10, wherein the rigid portion is made from cyclic olefin copolymer.

13. The system of claim 10, wherein the elastic portion is made from rubber, thermoplastic elastomer, butyl rubber, or polyisoprene elastomer.

14. The system of claim 10, wherein the elastic portion comprises:
an annular flap configured to be disposed adjacent a distal surface of the rigid portion;
a ring configured to support the annular flap; and
proximal and distal circumferential gaskets extending from an outer circumference of the ring,
wherein the proximal and distal circumferential gaskets are configured to form respective first and second fluid-tight seals with an inner surface of the injection system body.

15. The system of claim 14, wherein the rigid portion comprises an annular portion,
wherein the annular portion defines a port extending therethrough, and
wherein the port is configured to be removably closed by the annular flap of the elastic portion.

16. The system of claim 15, wherein the annular flap of the elastic portion is configured such that:
the annular flap is biased to removably close the port; and
the increased pressure in the proximal drug chamber relative to the distal drug chamber deforms the annular flap away from the port defined by the annular portion of the rigid portion, thereby opening the port.

17. The system of claim 14, wherein the annular portion defines a plurality of ports extending therethrough, and
wherein each of the plurality of ports is configured to be removably closed by the annular flap of the elastic portion.

18. The system of claim 17, wherein the annular flap of the elastic portion is configured such that:
the annular flap is biased to removably close each of the plurality of ports; and
the increased pressure in the proximal drug chamber relative to the distal drug chamber deforms the annular flap away from each of the plurality of ports defined by the annular portion of the rigid portion, thereby opening each of the plurality of ports.

19. The system of claim 10, wherein the rigid portion comprises a distally extending portion coupled to an annular portion, and
wherein the elastic portion has a radially inward circumferential surface defining a central port.

20. The system of claim 19, wherein the radially inward circumferential surface of the elastic portion is configured to form a fluid-tight seal around the distally extending portion of the rigid portion, thereby closing the central port in the elastic portion.

21. The system of claim 20, wherein the elastic portion is biased to close the central port therein with the distally extending portion of the rigid portion, and
wherein the increased pressure in the proximal drug chamber relative to the distal drug chamber deforms the elastic portion away from the distally extending portion of the rigid portion, removing the fluid-tight seal therearound, and opening the central port in the elastic portion.

22. The system of claim 19, wherein the distally extending portion of the rigid portion defines a circumferential groove configured to receive the radially inward circumferential surface of the elastic portion.

23. The system of claim 10, wherein the rigid portion defines a first port, and
wherein the elastic portion defines a second port.

24. The system of claim 23, wherein the fenestrated separator has a closed configuration and an open configuration,
wherein in the closed configuration, both the first and second ports are closed, and
wherein in the open configuration, both the first and second ports are open.

25. The system of claim 24, wherein in the open configuration a flow path fluidly couples the proximal and distal drug chambers through the first and second ports.

26. The system of claim 24, wherein the elastic portion is biased to close the first and second ports, thereby placing the fenestrated separator in the closed configuration.

27. The system of claim 24, wherein the fenestrated separator is configured to transform from the closed configuration to the open configuration with increased pressure in the proximal drug chamber relative to the distal drug chamber.

28.-41. (canceled)
